(19) United States
(12) Patent Application Publication
(10) Pub. No.: US 2008/0004295 A1
(43) Pub. Date: Jan. 3, 2008

(54) NOVEL COMPOUNDS

(76) Inventors: Paul Martin Gore, Stevenage (GB); Vipulkumar Kantibhai Patel, Stevenage (GB); Ann Louise Walker, Stevenage (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/548,343

(22) Filed: Oct. 11, 2006

(30) Foreign Application Priority Data
Oct. 13, 2005 (GB) 0520838.4
Jul. 6, 2006 (GB) 0613485.2
Sep. 15, 2006 (GB) 0618237.2

(51) Int. Cl. A61K 31/519; A61P 11/00; A61P 11/02; A61P 19/02; A61P 37/08; A61P 9/10; C07D 487/04

(52) U.S. Cl. 514/265.1; 544/280

(57) ABSTRACT

Pyrrolopyrimidine derivatives of formula (I) are inhibitors of Spleen Tyrosine kinase (Syk) and therefore of potential therapeutic benefit in the treatment of diseases and conditions associated with inappropriate Syk activity, in particular in the treatment of inflammatory and allergic diseases.

### FIELD OF THE INVENTION

The present invention relates to pyrrolopyrimidine derivatives, compositions and medicaments containing the same, as well as processes for the preparation and use of such compounds, compositions and medicaments. Such pyrrolopyrimidine derivatives are of potential therapeutic benefit in the treatment of diseases and conditions associated with inappropriate Syk activity, in particular in the treatment of inflammatory and allergic diseases.

### BACKGROUND TO THE INVENTION

Spleen Tyrosine Kinase (Syk) is a protein tyrosine kinase which has been described as a key mediator of immunoreceptor signalling in a host of inflammatory cells including mast cells, B-cells, macrophages and neutrophils.

These immunoreceptors, including Fc receptors and the B-cell receptor, are important for both allergic diseases and antibody-mediated autoimmune diseases and thus pharmacologically interfering with Syk could conceivably treat these disorders.

Allergic rhinitis and asthma are diseases associated with hypersensitivity reactions and inflammatory events involving a multitude of cell types including mast cells, eosinophils, T cells and dendritic cells. Following exposure to allergen, high affinity immunoglobulin receptors for IgE (FcεRI) and IgG (FcεRI) become cross-linked and activate downstream processes in mast cells and other cell types leading to the release of pro-inflammatory mediators and airway spasmogens. In the mast cell, for example, IgE receptor cross-linking by allergen leads to release of mediators including histamine from pre-formed granules, as well as the synthesis and release of newly synthesised lipid mediators including prostaglandins and leukotrienes.

Syk kinase is a non-receptor linked tyrosine kinase which is important in transducing the downstream cellular signals associated with cross-linking FcεR1 and or FcεR1 receptors, and is positioned early in the signalling cascade. In mast cells, for example, the early sequence of FcεR1 signalling following allergen cross-linking of receptor-IgE complexes involves first Lyn (a Src family tyrosine kinase) and then Syk. Inhibitors of Syk activity would therefore be expected to inhibit all downstream signalling cascades thereby alleviating the immediate allergic response and adverse events initiated by the release of pro-inflammatory mediators and spasmogens (Wong et al 2004, Expert Opin. Investig. Drugs (2004) 13 (7) 743-762).

Recently, it has been shown that the Syk kinase inhibitor R112 (Rigel), dosed intranasally in a phase I/II study for the treatment of allergic rhinitis, gave a statistically significant decrease in PGD₂, a key immune mediator that is highly correlated with improvements in allergic rhinorrhea, as well as being safe across a range of indicators, thus providing the first evidence for the clinical safety and efficacy of a topical Syk kinase inhibitor. (Meltzer, Eli O.; Berkowitz, Robert B.; Grossbard, Elliott B, Journal of Allergy and Clinical Immunology (2005), 115(4), 791-796). In a more recent phase II clinical trial for allergic rhinitis (Clinical Trials.gov Identifier NCT0015089), R112 was shown as having a lack of efficacy versus placebo.

Rheumatoid Arthritis (RA) is an auto-immune disease affecting approximately 1% of the population. It is characterised by inflammation of articular joints leading to debilitating destruction of bone and cartilage. Recent clinical studies with Rituximab, which causes a reversible B cell depletion, (J. C. W. Edwards et al 2004, New Eng. J. Med. 350: 2572-2581) have shown that targeting B cell function is an appropriate therapeutic strategy in auto-immune diseases such as RA. Clinical benefit correlates with a reduction in auto-reactive antibodies (or Rheumatoid Factor) and these studies suggest that B cell function and indeed auto-antibody production are central to the ongoing pathology in the disease.

Studies using cells from mice deficient in the Spleen Tyrosine Kinase (Syk) have demonstrated a non-redundant role of this kinase in B cell function. The deficiency in Syk is characterised by a block in B cell development (M. Turner et al 1995 Nature 379: 298-302 and Cheng et al 1995, Nature 378: 303-306). These studies, along with studies on mature B cells deficient in Syk (Kurasaki et al 2000, Immunol. Rev. 176:19-29), demonstrate that Syk is required for the differentiation and activation of B cells. Hence, inhibition of Syk in RA patients is likely block B cell function and thereby reduce Rheumatoid Factor production. In addition to the role of Syk in B cell function, and of further relevance to the treatment of RA, is the requirement for Syk activity in Fc receptor (FcR) signalling. FcR activation by immune complexes in RA has been suggested to contribute to the release of multiple pro-inflammatory mediators.

The present invention relates to novel pyrrolopyrimidine compounds, which are inhibitors of Syk kinase activity. Such pyrrolopyrimidine derivatives therefore have potential therapeutic benefit in the treatment of disorders associated with inappropriate Syk activity, in particular in the treatment and prevention of disease states mediated by Syk. Such disease states may include inflammatory, allergic and autoimmune diseases, for example, asthma, chronic obstructive pulmonary disease (COPD), adult respiratory distress syndrome (ARDS), ulcerative colitis, Crohns disease, bronchitis, dermatitis, allergic rhinitis, psoriasis, scleroderma, urticaria, rheumatoid arthritis, multiple sclerosis, cancer, HIV and lupus.

### BRIEF SUMMARY OF THE INVENTION

In one aspect of the present invention there is provided a compound of formula (I) or a salt or solvate thereof: 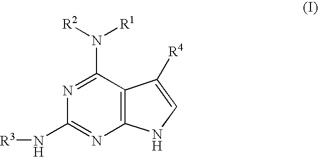
wherein:
R¹ is H or C₁₋₃ alkyl;
R² is C₁₋₆ alkyl, C₁₋₆-haloalkyl, C₃₋₇ cycloalkyl, or C₁₋₃ alkyleneC₃₋₇ cycloalkyl wherein each cycloalkyl may be substituted by one or more substituents independently selected from C₁₋₃ alkyl or halogen;
R³ is:
(a) a six membered heteroaryl group selected from 3-pyridinyl, 4-pyridinyl or 5-pyrimidinyl (each of which may be optionally substituted by one or more substituents independently selected from OH, ═O, C₁₋₃ alkyl, NHCOC₁₋₁₃ alkyl, C₁₋₆ alkoxy, COC₁₋₁₆ alkyl, C₀₋₃ alkylene COOC₁₋₃ alkyl);
(b) a group 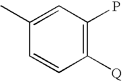
wherein P and Q together form a 5-7 membered carbocyclic, heterocyclic or heteroaryl ring, which rings may be optionally substituted by one or more substituents independently selected from; on each carbon by up two C₁₋₃alkyl groups or fluorines or by ═O or by OH, C₁₋₃alkoxy, C₁₋₃haloalkyl, C₀₋₃ alkyleneNR⁵R⁶, on each nitrogen by C₁₋₃alkyl, COC₁₋₃alkyl, C₁₋₃alkyleneC₃₋₇cycloalkyl, phenyl (optionally substituted by fluorine) or C₀₋₃ alkyleneNR⁵R⁶ or on sulphur by ═O or (═O)₂;
R⁵ and R⁶ are independently H or C₁₋₃ alkyl;
(c) a group 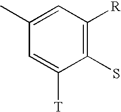
wherein one of R, S and T is H and the remaining substituents are independently selected from:
H, C₁₋₆alkyl, C₁₋₆haloalkyl, C₁₋₆alkoxy, OH, C₁₋₆ hydroxyalkyl, CN, C₃₋₇cycloalkyl, Ophenyl, OCH₂phenyl, halogen, COOR⁷, C₁₋₃alkyleneCOOR⁷, XNR⁸R⁹, XCONR⁸R⁹, XSO₂NR⁸R⁹, NR⁷COC₁₋₆alkyl, NR⁷SO₂C₁₋₆alkyl, OCH₂CONR⁸R⁹, SO₂C₁₋₃alkyl, a monocyclic heteroaryl group (optionally substituted by methyl);
R⁷ is H or —C₁₋₃ alkyl;
X is a bond or C₁₋₃alkylene;
R⁸ and R⁹ are independently H, C₁₋₆alkyl, C₁₋₆haloalkyl, C₁₋₆hydroxyalkyl, C₃₋₇cycloalkyl, C₁₋₃ alkyleneC₃₋₇ cycloalkyl, phenyl (optionally substituted by one or more substitutents independently selected from halogen, —C₁₋₃ alkyl, CN, or SO₂CF₃), C₁₋₃ alkylenephenyl, C₁₋₃ alkyleneOC₁₋₃ alkyl; or
R⁸ and R⁹ together with N to which they are joined form a 4-, 5- or 6 membered heterocyclic group, optionally containing a further heteroatom selected from O, S, or N and optionally substituted by on each carbon by up to two C₁₋₆ alkyl or halogen, or by ═O or C₁₋₆ alkoxy, on any optional nitrogen by C₁₋₆alkyl, COC₁₋₃alkyl or COOC₁₋₁₆ alkyl and on any optional sulphur by ═O, (═O)₂;
R⁴ is H or —C₁₋₃ alkyl.

In a further aspect of the present invention, there is provided a pharmaceutical composition comprising a compound of formula (I), or a salt or solvate, thereof and one or more of pharmaceutically acceptable carriers, diluents and excipients.

In a further aspect of the present invention, there is provided a compound of formula (I), or a salt or solvate, thereof for use in therapy.

In a further aspect of the present invention, there is provided a compound of formula (I) or a salt or solvate thereof for use in the treatment of a disease or condition mediated by inappropriate Syk activity.

In a further aspect of the present invention there is provided the use of a compound of formula (I) or a salt or solvate thereof in the manufacture of a medicament for use in the treatment of a disease or condition mediated by inappropriate Syk activity.

### DETAILED DESCRIPTION OF THE INVENTION

As used herein, the term “effective amount” means that amount of a drug or pharmaceutical agent that will elicit the biological or medical response of a tissue, system, animal or human that is being sought, for instance, by a researcher or clinician.

Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein the term “alkyl” refers to a straight- or branched-chain hydrocarbon radical having the specified number of carbon atoms. As used herein, the terms “C₁-C₃alkyl” and “C₁-C₆alkyl” refer to an alkyl group, as defined above, containing at least 1, and at most 3 or 6 carbon atoms respectively. Examples of “alkyl” as used herein include, but are not limited to, methyl, ethyl, n-propyl, isopropyl, n-butyl, isobutyl, t-butyl, n-pentyl, isopentyl, and the like.

As used herein, the term “alkylene” refers to a straight or branched chain divalent hydrocarbon radical having the specified number of carbon atoms. As used herein, the terms “C₁-C₃ alkylene” and “C₁-C₆ alkylene” refer to an alkylene group, as defined above, which contains at least 1, and at most 3 or 6, carbon atoms respectively. Examples of “alkylene” as used herein include, but are not limited to, methylene, ethylene, n-propylene, n-butylene, and the like.

As used herein, the term “halogen” refers to fluorine (F), chlorine (Cl), bromine (Br), or iodine (I) and the term “halo” refers to the halogen radicals: fluoro (—F), chloro (—Cl), bromo (—Br), and iodo (—I).

As used herein, the term “haloalkyl” refers to an alkyl group as defined above, substituted with at least one halo group, halo being as defined herein. Examples of such branched or straight chained haloalkyl groups useful in the present invention include, but are not limited to, methyl, ethyl, propyl, isopropyl, isobutyl and n-butyl substituted independently with one or more halos, e.g., fluoro, chloro, bromo and iodo.

As used herein, the term “cycloalkyl” refers to a non-aromatic cyclic hydrocarbon ring containing the specified number of carbon atoms. In a like manner the term “C₃-C₇ cycloalkyl” refers to a non-aromatic cyclic hydrocarbon ring having from 3 to 7 carbon atoms. Exemplary “cycloalkyl” groups useful in the present invention include, but are not limited to, cyclopropyl, cyclobutyl, cyclopentyl, cyclohexyl and cycloheptyl.

As used herein, the term “carbocyclic” refers to a non-aromatic ring containing carbon and hydrogen atoms, being saturated or having one or more degrees of unsaturation.

As used herein, the term “heterocyclic” or the term “heterocyclyl” refers to a non-aromatic heterocyclic ring, being saturated or having one or more degrees of unsaturation, containing one or more heteroatom substitutions selected from S, S(O), S(O)₂, O, or N, and having the specified number of ring members.

As used herein, the term “alkoxy” refers to the group RₐO, where Rₐ is alkyl as defined above and the terms “C₁-C₃ alkoxy” and “C₁-C₆ alkoxy” refer to an alkoxy group as defined herein wherein the alkyl moiety contains at least 1, and at most 3 or 6, carbon atoms. Exemplary “C₁-C₃ alkoxy” and “C₁-C₆ alkoxy” groups useful in the present invention include, but are not limited to, methoxy, ethoxy, n-propoxy, isopropoxy, n-butoxy, and t-butoxy.

As used herein, the term “haloalkoxy” refers to the group RₐO—, where Rₐ is haloalkyl as defined above and the term “C₁-C₆ haloalkoxy” refers to a haloalkoxy group as defined herein wherein the haloalkyl moiety contains at least 1, and at most 6, carbon atoms. Exemplary C₁-C₆ haloalkoxy groups useful in the present invention include, but are not limited to, trifluoromethoxy.

As used herein the term “hydroxy” refers to the group —OH.

The term “heteroaryl”, unless otherwise specified, refers to aromatic monocyclic groups and fused bicyclic aromatic rings, having the specified number of ring members (e.g. carbon and heteratoms N, O, and/or S) and containing 1, 2, 3 or 4 heteroatoms selected from N, O and S. Examples of particular heteroaryl groups include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiopene, benzazepine, benzimidazole, benzoimidazole, indole, oxindole and indazole.

As used herein, the term “hydroxyalkyl” refers to an alkyl group as defined above substituted with at least one hydroxy, hydroxy being as defined herein. Examples of branched or straight chained “C₁-C₆ hydroxyalkyl” groups useful in the present invention include, but are not limited to, methyl, ethyl, propyl, isopropyl, isobutyl and n-butyl substituted independently with one or more hydroxy groups.

As used herein, the term “optionally” means that the subsequently described event(s) may or may not occur, and includes both event(s), which occur, and events that do not occur.

As used herein, the term “substituted” refers to substitution with the named substituent or substituents, multiple degrees of substitution being allowed unless otherwise stated.

The term “Syk inhibitor”, is used to mean a compound which inhibits the Syk receptor.

The term “Syk mediated disease” or a “disorder or disease or condition mediated by inappropriate Syk activity” is used to mean any disease state mediated or modulated by Syk kinase mechanisms. Such disease states may include inflammatory, allergic and autoimmune diseases, for example, asthma, chronic obstructive pulmonary disease (COPD), adult respiratory distress syndrome (ARDs), ulcerative colitis, Crohns disease, bronchitis, dermatitis, allergic rhinitis, psorasis, scleroderma, urticaria, rheumatoid arthritis, multiple sclerosis, cancer, HIV and lupus, in particular, asthma, chronic obstructive pulmonary disease (COPD), adult respiratory distress syndrome (ARDs), allergic rhinitis and rheumatoid arthritis.

As used herein, “a compound of the invention” means a compound of formula (I) or a salt, solvate or physiologically functional derivative thereof.

As used herein, the term “solvate” refers to a complex of variable stoichiometry formed by a solute (in this invention, a compound of formula (I), or a salt thereof) and a solvent. Such solvents for the purpose of the invention may not interfere with the biological activity of the solute. Examples of suitable solvents include, but are not limited to, water, acetone, methanol, ethanol and acetic acid. Preferably the solvent used is a pharmaceutically acceptable solvent. Examples of suitable pharmaceutically acceptable solvents include water, ethanol and acetic acid. Most preferably the solvent is water.

The compounds of formula (I) may have the ability to crystallize in more than one form, a characteristic, which is known as polymorphism, and it is understood that such polymorphic forms (“polymorphs”) are within the scope of formula (I). Polymorphism generally can occur as a response to changes in temperature or pressure or both and can also result from variations in the crystallization process. Polymorphs can be distinguished by various physical characteristics known in the art such as x-ray diffraction patterns, solubility and melting point.

Certain of the compounds described herein may contain one or more chiral atoms, or may otherwise be capable of existing as two enantiomers. Accordingly, the compounds of this invention include mixtures of enantiomers as well as purified enantiomers or enantiomerically enriched mixtures. Also included within the scope of the invention are the individual isomers of the compounds represented by formula (I) above as well as any wholly or partially equilibrated mixtures thereof. The present invention also covers the individual isomers of the compounds represented by the formulas above as mixtures with isomers thereof in which one or more chiral centres are inverted.

It is also noted that the compounds of Formula (I) may form tautomers. It is understood that all tautomers and mixtures of tautomers of the compounds of the present invention are included within the scope of the compounds of the present invention.

In one embodiment, R¹ represents H or methyl. In a further embodiment R¹ represents H.

In one embodiment, R² represents C₁₋₃ alkyl, for example 1-methylethyl. In a further embodiment, R² represents C₁₋₃ haloalkyl, for example 1-trifluoroethyl.

In one embodiment, R¹ represents H and R² is C₁₋₃alkyl, for example 1-methylethyl. In a further embodiment, R¹ represents H and R² is C₁₋₃ haloalkyl, for example 1-trifluoroethyl.

In one embodiment, R⁴ is H or CH₃. In a further embodiment, R⁴ is H.

In one embodiment, R³ is a group 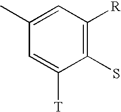
wherein one of R, S and T is H and the remaining substituents are independently selected from:
H, C₁₋₆alkyl, C₁₋₆haloalkyl, C₁₋₆alkoxy, OH, C₁₋₆ hydroxyalkyl, CN, C₃₋₇cycloalkyl, Ophenyl, OCH₂phenyl, halogen, COOR⁷, C₁₋₃alkyleneCOOR⁷, XNR⁸R⁹, XCONR⁸R⁹, XSO₂NR⁸R⁹, NR⁷COC₁₋₆alkyl, NR⁷SO₂C₁₋₆alkyl, OCH₂CONR⁸R⁹, SO₂C₁₋₃alkyl, a monocyclic heteroaryl group (optionally substituted by methyl);
R⁷ is H or —C₁₋₃ alkyl;
X is a bond or C₁₋₃alkylene; and
R³ and R⁹ are as hereinbefore defined.

In a further embodiment, R³ is a group: 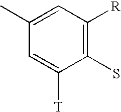
wherein R is H, and S and T are independently selected from:
H, C₁₋₆alkyl, C₁₋₆haloalkyl, C₁₋₆alkoxy, OH, C₁₋₆ hydroxyalkyl, CN, C₃₋₇cycloalkyl, Ophenyl, OCH₂phenyl, halogen, COOR⁷, C₁₋₃alkyleneCOOR⁷, XNR⁸R⁹, XCONR⁸R⁹, XSO₂NR⁸R⁹, NR⁷COC₁₋₆alkyl, NR⁷SO₂C₁₋₆alkyl, OCH₂CONR⁸R⁹, SO₂C₁₋₃alkyl, a monocyclic heteroaryl group (optionally substituted by methyl);
X is a bond or C₁₋₃alkylene; and
R⁷, R³ and R⁹ are as hereinbefore defined.

In a further embodiment, R³ is a group: 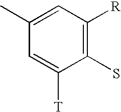
wherein R is H, S is XCONR⁸R⁹, and X is a bond, and T is hydrogen or halogen;
and R³ and R⁹ are as hereinbefore defined.

In a further embodiment, R³ is a group: 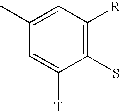
wherein R and T is each hydrogen and S is CONR⁸R⁹;
and R³ and R⁹ are as hereinbefore defined.

In one embodiment, R⁸ and R⁹ is each is hydrogen.

In one embodiment, R⁸ is hydrogen and R⁹ is C₁₋₆alkyl, C₁₋₆haloalkyl, C₃₋₇cycloalkyl, or C₁₋₃ alkyleneC₃₋₇ cycloalkyl, preferably n-propyl

In one embodiment, R⁸ is C₁₋₆alkyl, C₁₋₆haloalkyl, C₃₋₇cycloalkyl, C₁₋₃alkyleneC₃₋₇ cycloalkyl and R⁹ is C₁₋₆alkyl, C₁₋₆haloalkyl, C₃₋₇cycloalkyl, C₁₋₃alkyleneC₃₋₇ cycloalkyl.

In one embodiment, R³ and R⁹, together with N to which they are joined form a 4-, 5- or 6 membered heterocyclic group, optionally containing a further heteroatom selected from O, S, or N, and optionally substituted on any optional nitrogen by C₁₋₆alkyl and on any optional sulphur by ═O, or (═O)₂.

In a further embodiment, there is provided a compound of formula (IA) or a salt or solvate thereof: 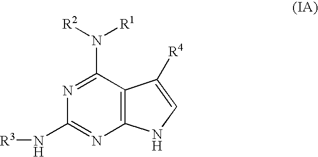
wherein:
R¹ represents H;
R² is C₁₋₃ haloalkyl;
R³ is a group: 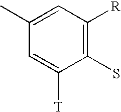
wherein R and T is each hydrogen, and S is CONR⁸R⁹;
R⁸ is hydrogen and R⁹ is C₁₋₆alkyl, C₁₋₆haloalkyl, C₃₋₇cycloalkyl, C₁₋₃ alkyleneC₃₋₇ cycloalkyl, preferably n-propyl; or
R⁸ is C₁₋₆alkyl, C₁₋₆haloalkyl, C₃₋₇cycloalkyl, C₁₋₃alkyleneC₃₋₇ cycloalkyl and R⁹ is C₁₋₆alkyl, C₁₋₆haloalkyl, C₃₋₇cycloalkyl, C₁₋₃alkyleneC₃₋₇ cycloalkyl, or
R⁸ and R⁹; together with N to which they are joined form a 4-, 5- or 6 membered heterocyclic group, optionally containing a further heteroatom selected from O, S, or N, and optionally substituted on any optional nitrogen by C₁₋₆alkyl and on any optional sulphur by ═O, (=°)₂, and
R⁴ is H.

It will be appreciated that formula (IA) may also be expressed as formula (IB): 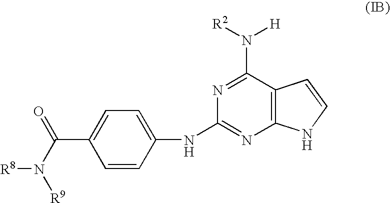
when values for R¹, R³ and R⁴ are inserted.

Whilstst the embodiments for each variable have generally been listed above separately for each variable, this invention also includes those compounds in which several or each embodiment in formula (I) is selected from each of the embodiments listed above. Therefore, this invention is intended to include all combinations of embodiments for each variable.

Specific examples of compounds of the present invention include Examples 1-52 as described in the Examples section below, in particular:
N-Propyl-4-({4-[(2,2,2-trifluoroethyl)amino]-1H-pyrrolo[2,3-d]pyrimidin-2-yl}amino)benzamide;
   or a pharmaceutically acceptable salt or solvate thereof

The compounds of the present invention may be in the form of and/or may be administered as a pharmaceutically acceptable salt. For a review on suitable salts see Berge et al, J. Pharm. Sci. 1977, 66, 1-19.

Typically, the salts of the present invention are pharmaceutically acceptable salts. Salts encompassed within the term “pharmaceutically acceptable salts” refer to non-toxic salts of the compounds of this invention.

Suitable pharmaceutically acceptable salts can include acid or base additions salts.

A pharmaceutically acceptable acid addition salt can be formed by reaction of a compound of formula (I) with a suitable inorganic or organic acid (such as hydrobromic, hydrochloric, sulfuric, nitric, phosphoric, succinic, maleic, formic, acetic, propionic, fumaric, citric, tartaric, lactic, benzoic, salicylic, glutamic, aspartic, p-toluenesulfonic, benzenesulfonic, methanesulfonic, ethanesulfonic, naphthalenesulfonic such as 2-naphthalenesulfonic, or hexanoic acid), optionally in a suitable solvent such as an organic solvent, to give the salt which is usually isolated, for example, by crystallisation and filtration. A pharmaceutically acceptable acid addition salt of a compound of formula (I) can comprise or be, for example, a hydrobromide, hydrochloride, sulfate, nitrate, phosphate, succinate, maleate, formarate, acetate, propionate, fumarate, citrate, tartrate, lactate, benzoate, salicylate, glutamate, aspartate, p-toluenesulfonate, benzenesulfonate, methanesulfonate, ethanesulfonate, naphthalenesulfonate (e.g. 2-naphthalenesulfonate) or hexanoate salt.

Other, non-pharmaceutically acceptable, salts, e.g. oxalates or trifluoroacetates, may also be used, for example, in the isolation of compounds of the invention, and are included within the scope of this invention.

The invention includes within its scope all possible stoichiometric and non-stoichiometric forms of the compounds of formula (I).

The compounds of formula (I) and salts, solvates and physiologically functional derivatives thereof are believed to be inhibitors of Syk activity, and thus be potentially useful in the treatment of diseases and conditions associated with inappropriate Syk activity.

The invention thus provides compounds of formula (I) and salts, solvates and physiologically functional derivatives thereof for use in therapy, and particularly in the treatment of diseases and conditions mediated by inappropriate Syk activity.

The inappropriate Syk activity referred to herein is any Syk activity that deviates from the normal Syk activity expected in a particular mammalian subject. Inappropriate Syk activity may take the form of, for instance, an abnormal increase in activity, or an aberration in the timing and or control of Syk activity. Such inappropriate activity may result then, for example, from overexpression or mutation of the protein kinase leading to inappropriate or uncontrolled activation.

In a further embodiment, the present invention is directed to methods of regulating, modulating, or inhibiting Syk for the prevention and/or treatment of disorders related to unregulated Syk activity.

In a further embodiment, the present invention provides a method of treatment of a mammal suffering from a disorder mediated by Syk activity, which includes administering to said subject an effective amount of a compound of formula (I) or a pharmaceutically acceptable salt, solvate, or a physiologically functional derivative thereof.

In a further embodiment, the present invention provides for the use of a compound of formula (I), or a pharmaceutically acceptable salt or solvate thereof, or a physiologically functional derivative thereof, in the preparation of a medicament for the treatment of a disorder mediated by Syk activity.

In a further embodiment, the disease or condition mediated by inappropriate Syk activity is rheumatoid arthritis.

In a further embodiment, the disease or condition mediated by inappropriate Syk activity is allergic rhinitis.

In a further embodiment, the disease or condition mediated by inappropriate Syk activity is chronic obstructive pulmonary disease (COPD).

In a further embodiment, the disease or condition mediated by inappropriate Syk activity is adult respiratory distress syndrome (ARDs).

While it is possible that, for use in therapy, a compound of formula (I), as well as salts, solvates and physiological functional derivatives thereof, may be administered as the raw chemical, it is possible to present the active ingredient as a pharmaceutical composition. Accordingly, the invention further provides a pharmaceutical composition, which comprises a compound of formula (I) and salts, solvates and physiological functional derivatives thereof, and one or more pharmaceutically acceptable carriers, diluents, or excipients. The compounds of the formula (I) and salts, solvates and physiological functional derivatives thereof, are as described above. The carrier(s), diluent(s) or excipient(s) must be acceptable in the sense of being compatible with the other ingredients of the formulation and not deleterious to the recipient thereof. In accordance with another aspect of the invention there is also provided a process for the preparation of a pharmaceutical composition including admixing a compound of the formula (I), or salts, solvates and physiological functional derivatives thereof, with one or more pharmaceutically acceptable carriers, diluents or excipients.

Pharmaceutical compositions of the present invention may be presented in unit dose forms containing a predetermined amount of active ingredient per unit dose. Such a unit may contain, for example, 5 μg to 1 g, preferably 1 mg to 700 mg, more preferably 5 mg to 100 mg of a compound of the formula (I), depending on the condition being treated, the route of administration and the age, weight and condition of the patient. Such unit doses may therefore be administered more than once a day. Preferred unit dosage compositions are those containing a daily dose or sub-dose (for administration more than once a day), as herein above recited, or an appropriate fraction thereof, of an active ingredient. Furthermore, such pharmaceutical compositions may be prepared by any of the methods well known in the pharmacy art.

Pharmaceutical compositions of the present invention may be adapted for administration by any appropriate route, for example by the oral (including buccal or sublingual), inhaled, or nasalroute. Such compositions may be prepared by any method known in the art of pharmacy, for example by bringing into association the active ingredient with the carrier(s) or excipient(s).

In a further embodiment, the present invention provides a pharmaceutical composition adapted for administration by the oral route, for treating, for example, rheumatoid arthritis.

In a further embodiment, the present invention provides a pharmaceutical composition adapted for administration by the nasal route, for treating, for example, allergic rhinitis.

In a further embodiment, the present invention provides a pharmaceutical composition adapted for administration by the inhaled route, for treating, for example, COPD or ARDS.

Pharmaceutical compositions of the present invention which are adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The formulation can also be prepared to prolong or sustain the release, for example, by coating or embedding particulate material in polymers, wax or the like.

The compounds of formula (I), and salts, solvates and physiological functional derivatives thereof, can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The compounds of formula (I) and salts, solvates and physiological functional derivatives thereof may also be delivered by the use of monoclonal antibodies as individual carriers to which the compound molecules are coupled. The compounds may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Dosage forms for inhaled administration may conveniently be formulated as aerosols or dry powders.

For compositions suitable and/or adapted for inhaled administration, it is preferred that the compound or salt of formula (I) is in a particle-size-reduced form, and more preferably the size-reduced form is obtained or obtainable by micronisation. The preferable particle size of the size-reduced (e.g. micronised) compound or salt or solvate is defined by a D50 value of about 0.5 to about 10 microns (for example as measured using laser diffraction).

Aerosol formulations, e.g. for inhaled administration, can comprise a solution or fine suspension of the active substance in a pharmaceutically acceptable aqueous or non-aqueous solvent. Aerosol formulations can be presented in single or multidose quantities in sterile form in a sealed container, which can take the form of a cartridge or refill for use with an atomising device or inhaler. Alternatively the sealed container may be a unitary dispensing device such as a single dose nasal inhaler or an aerosol dispenser fitted with a metering valve (metered dose inhaler) which is intended for disposal once the contents of the container have been exhausted.

Where the dosage form comprises an aerosol dispenser, it preferably contains a suitable propellant under pressure such as compressed air, carbon dioxide or an organic propellant such as a hydrofluorocarbon (HFC). Suitable HFC propellants include 1,1,1,2,3,3,3-heptafluoropropane and 1,1,1,2-tetrafluoroethane. The aerosol dosage forms can also take the form of a pump-atomiser. The pressurised aerosol may contain a solution or a suspension of the active compound. This may require the incorporation of additional excipients e.g. co-solvents and/or surfactants to improve the dispersion characteristics and homogeneity of suspension formulations. Solution formulations may also require the addition of co-solvents such as ethanol. Other excipient modifiers may also be incorporated to improve, for example, the stability and/or taste and/or fine particle mass characteristics (amount and/or profile) of the formulation.

For pharmaceutical compositions suitable and/or adapted for inhaled administration, it is preferred that the pharmaceutical composition is a dry powder inhalable composition. Such a composition can comprise a powder base such as lactose, glucose, trehalose, mannitol or starch, the compound of formula (I) or salt or solvate thereof (preferably in particle-size-reduced form, e.g. in micronised form), and optionally a performance modifier such as L-leucine or another amino acid, and/or metals salts of stearic acid such as magnesium or calcium stearate. Preferably, the dry powder inhalable composition comprises a dry powder blend of lactose and the compound of formula (I) or salt thereof. The lactose is preferably lactose hydrate e.g. lactose monohydrate and/or is preferably inhalation-grade and/or fine-grade lactose. Preferably, the particle size of the lactose is defined by 90% or more (by weight or by volume) of the lactose particles being less than 1000 microns (micrometres) (e.g. 10-1000 microns e.g. 30-1000 microns) in diameter, and/or 50% or more of the lactose particles being less than 500 microns (e.g. 10-500 microns) in diameter. More preferably, the particle size of the lactose is defined by 90% or more of the lactose particles being less than 300 microns (e.g. 10-300 microns e.g. 50-300 microns) in diameter, and/or 50% or more of the lactose particles being less than 100 microns in diameter. Optionally, the particle size of the lactose is defined by 90% or more of the lactose particles being less than 100-200 microns in diameter, and/or 50% or more of the lactose particles being less than 40-70 microns in diameter. Most importantly, it is preferable that about 3 to about 30% (e.g. about 10%) (by weight or by volume) of the particles are less than 50 microns or less than 20 microns in diameter. For example, without limitation, a suitable inhalation-grade lactose is E9334 lactose (10% fines) (Borculo Domo Ingredients, Hanzeplein 25, 8017 J D Zwolle, Netherlands).

Optionally, in particular for dry powder inhalable compositions, a pharmaceutical composition for inhaled administration can be incorporated into a plurality of sealed dose containers (e.g. containing the dry powder composition) mounted longitudinally in a strip or ribbon inside a suitable inhalation device. The container is rupturable or peel-openable on demand and the dose of e.g. the dry powder composition can be administered by inhalation via the device such as the DISKUS™ device, marketed by GlaxoSmithKline. The DISKUS™ inhalation device is for example described in GB 2242134 A, and in such a device at least one container for the pharmaceutical composition in powder form (the container or containers preferably being a plurality of sealed dose containers mounted longitudinally in a strip or ribbon) is defined between two members peelably secured to one another; the device comprises: a means of defining an opening station for the said container or containers; a means for peeling the members apart at the opening station to open the container; and an outlet, communicating with the opened container, through which a user can inhale the pharmaceutical composition in powder form from the opened container.

Dosage forms for nasal administration may conveniently be formulated as aerosols, solutions, drops, gels or dry powders.

Pharmaceutical compositions adapted for administration by inhalation include fine particle dusts or mists, which may be generated by means of various types of metered, dose pressurised aerosols, nebulizers or insufflators.

For pharmaceutical compositions suitable and/or adapted for intranasal administration, the compound of formula (I) or a pharmaceutically acceptable salt or solvate thereof may be formulated as a fluid formulation for delivery from a fluid dispenser. Such fluid dispensers may have, for example, a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO-A-2005/044354, the entire content of which is hereby incorporated herein by reference. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. A particularly preferred fluid dispenser is of the general type illustrated in FIGS. 30-40 of WO-A-2005/044354.

It will be appreciated that when the compound of the present invention is administered in combination with other therapeutic agents normally administered by the inhaled, intravenous, oral or intranasal route, that the resultant pharmaceutical composition may be administered by the same routes.

It should be understood that in addition to the ingredients particularly mentioned above, the compositions may include other agents conventional in the art having regard to the type of formulation in question, for example those suitable for oral administration may include flavouring agents.

A therapeutically effective amount of a compound of the present invention will depend upon a number of factors including, for example, the age and weight of the animal, the precise condition requiring treatment and its severity, the nature of the formulation, and the route of administration, and will ultimately be at the discretion of the attendant physician or veterinarian However, an effective amount of a compound of formula (I) for the treatment of diseases or conditions associated with inappropriate Syk activity, will generally be in the range of 5 μg to 100 mg/kg body weight of recipient (mammal) per day and more usually in the range of 5 μg to 10 mg/kg body weight per day. This amount may be given in a single dose per day or more usually in a number (such as two, three, four, five or six) of sub-doses per day such that the total daily dose is the same. An effective amount of a salt or solvate, thereof, may be determined as a proportion of the effective amount of the compound of formula (I) per se.

Compounds of the present invention, and their salts and solvates, and physiologically functional derivatives thereof, may be employed alone or in combination with other therapeutic agents for the treatment of diseases and conditions associated with inappropriate tyrosine and serine/threonine kinase activity. Combination therapies according to the present invention thus comprise the administration of at least one compound of formula (I) or a pharmaceutically acceptable salt or solvate thereof, or a physiologically functional derivative thereof, and the use of at least one other pharmaceutically active agent. Preferably, combination therapies according to the present invention comprise the administration of at least one compound of formula (I) or a pharmaceutically acceptable salt or solvate thereof, or a physiologically functional derivative thereof, and at least one other pharmaceutically active agent. The compound(s) of formula (I) and the other pharmaceutically active agent(s) may be administered together or separately and, when administered separately this may occur simultaneously or sequentially in any order. The amounts of the compound(s) of formula (I) and the other pharmaceutically active agent(s) and the relative timings of administration will be selected in order to achieve the desired combined therapeutic effect.

Compounds of the present invention, and their salts and solvates, and physiologically functional derivatives thereof, may also be used in combination with other classes of therapeutic agents which are known in the art. Representative classes of agents for use in such combinations include, for treating asthma, anti-inflammatory steroids (in particular corticosteroids), topical glucocorticoid agonists, PDE4 inhibitors, IKK2 inhibitors, A2a agonists, β₂-adrenoreceptor agonists (including both slow acting and long acting β₂-adrenoreceptor agonists), alpha 4 integrin inhibitors, and anti-muscarinics, and, for treating allergies, the foregoing agents, as well as H1 and H1/H3 antagonists. Representative agents for use in combination therapy for treating severe asthma include topically acting p38 inhibitors, and IKK2 inhibitors.

Anti-inflammatory corticosteroids are well known in the art. Representative examples include fluticasone propionate (e.g. see U.S. Pat. No. 4,335,121), beclomethasone 17-propionate ester, beclomethasone 17,21-dipropionate ester, dexamethasone or an ester thereof, mometasone or an ester thereof (e.g. mometasone furoate), ciclesonide, budesonide, and flunisolide. Further examples of anti-inflammatory corticosteroids are described in WO 02/12266 A1 (Glaxo Group Ltd), in particular, the compounds of Example 1 (6α,9α-difluoro-17α-[(2-furanylcarbonyl)oxy]-10-hydroxy-16α-methyl-3-oxo-androsta-1,4-diene-17β-carbothioic acid S-fluoromethyl ester) and Example 41 (6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester), or a pharmaceutically acceptable salt thereof.

Examples of β₂-adrenoreceptor agonists include salmeterol (e.g. as racemate or a single enantiomer such as the R-enantiomer), salbutamol, formoterol, salmefamol, fenoterol or terbutaline and salts thereof, for example the xinafoate salt of salmeterol, the sulphate salt or free base of salbutamol or the fumarate salt of formoterol. Long-acting β₂-adrenoreceptor agonists are preferred, especially those having a therapeutic effect over a 24 hour period such as salmeterol or formoterol.

Examples of anti-histamines include azelastine, levocabastine, olopatidine, methapyrilene, loratadine, cetirizine, desloratadine or fexofenadine.

Examples of anticholinergic compounds include muscarinic (M) receptor antagonists, in particular M₁, M₂, M₁/M₂, or M₃ receptor antagonists, in particular a (selective) M₃ receptor antagonist. Examples of anticholinergic compounds are described in WO 03/011274 A2 and WO 02/069945 A2/US 2002/0193393 A1 and US 2002/052312 A1. Examples of muscarinic M3 antagonists include ipratropium bromide, oxitropium bromide or tiotropium bromide.

Representative PDE4 or mixed PDE3/4 inhibitors that may be used in combination with compounds of the invention include AWD-12-281 (Elbion), PD-168787 (Pfizer), roflumilast, and cilomilast (GlaxoSmithKline). Further examples of PDE4 inhibitors are described in WO 2004/103998 (Glaxo Group Ltd).

The present invention also provides for so-called “triple combination” therapy, comprising a compound of formula (I) or a pharmaceutically acceptable salt thereof together with β₂-adrenoreceptor agonist and an anti-inflammatory corticosteroid. Preferably this combination is for treatment and/or prophylaxis of asthma, COPD or allergic rhinitis. The β₂-adrenoreceptor agonist and/or the anti-inflammatory corticosteroid can be as described above and/or as described in WO 03/030939 A1. A representative example of such a “triple” combination comprises a compound of formula (I) or a pharmaceutically acceptable salt thereof, salmeterol or a pharmaceutically acceptable salt thereof (e.g. salmeterol xinafoate) and fluticasone propionate.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

The combinations referred to above may conveniently be presented for use in the form of a pharmaceutical composition and thus pharmaceutical compositions comprising a combination as defined above together with a pharmaceutically acceptable diluent or carrier represent a further aspect of the invention. These combinations are of particular interest in respiratory diseases and are conveniently adapted for inhaled or intranasal delivery.

Rheumatoid arthritis (RA) is a further inflammatory disease where combination therapy may be contemplated. Thus in a further aspect, the present invention provides a compound of formula (I) or a salt or solvate thereof in combination with a further therapeutic agent useful in the treatment of rheumatoid arthritis, said combination being useful for the treatment of rheumatoid arthritis.

The compound and pharmaceutical compositions according to the invention may be used in combination with or include one or more other therapeutic agents, for example selected from NSAIDS, corticosteroids, COX-2 inhibitors, cytokine inhibitors, anti-TNF agents, inhibitors of oncostatin M, anti-malarials, immunosuppressivess and cytostatics

Two classes of medication are contemplated for the treatment of RA, these may be classified as “fast acting” and “slow acting” or “second line” drugs (also referred to as Disease Modifying Antirheumatic Drugs or DMARDS). The first line drugs such as typical NSAIDs (e.g. aspirin, ibuprofen, naproxen, etodolac), corticosteroids (e.g. prednisone). Second line drugs include COX-2 inhibitors and anti-TNF agents. Examples of COX-2 inhibitors are celecoxib (Celebrex), etoricoxib and rofecoxib (Vioxx).

Anti-TNF agents include infliximab (Remicade), etanercept (Enbrel) and adalimumab (Humira). Other “biological” treatments include anakinra (Kineret), Rituximab, Lymphostat-B, BAFF/APRIL inhibitors and CTLA-4-1 g or mimetics thereof. Other cytokine inhibitors include leflunomide (Arava). Further second line drugs include gold preparations (Auranofin (Ridaura tablets) or Aurothiomalate (Myocrisin injection)), medicines used for malaria: (Hydroxychloroquine (Plaquenil)), medicines that suppress the immune system (Azathioprine (Imuran, Thioprine), methotrexate (Methoblastin, Ledertrexate, Emthexate), cyclosporin (Sandimmun, Neoral)), Cyclophosphamide (Cycloblastin), Cytoxan, Endoxan), D-Penicillamine (D-Penamine), Sulphasalazine (Salazopyrin), nonsteroidal anti inflammatory drugs (including aspirin and ibuprofen).

The individual compounds of such combinations may be administered either sequentially or simultaneously in separate or combined pharmaceutical compositions. Preferably, the individual compounds will be administered simultaneously in a combined pharmaceutical composition. Appropriate doses of known therapeutic agents will be readily appreciated by those skilled in the art.

The compounds of this invention may be made by a variety of methods, including standard chemistry. Any previously defined variable will continue to have the previously defined meaning unless otherwise indicated. Illustrative general synthetic methods are set out below and then specific compounds of the invention are prepared in the Working Examples.

Compounds of general formula (I) may be prepared by methods known in the art of organic synthesis as set forth in part by the following synthesis schemes. In all of the schemes described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art. The selection of processes as well as the reaction conditions and order of their execution shall be consistent with the preparation of compounds of Formula (I). Those skilled in the art will recognize if a stereocenter exists in compounds of Formula (I). Accordingly, the present invention includes both possible stereoisomers and includes not only racemic compounds but the individual enantiomers as well. When a compound is desired as a single enantiomer, it may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994). 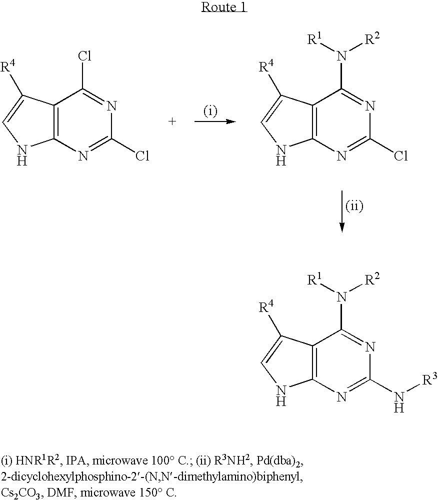 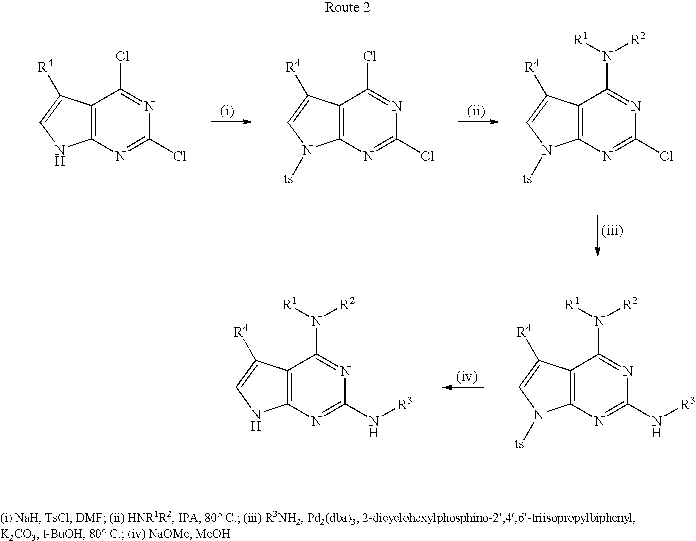 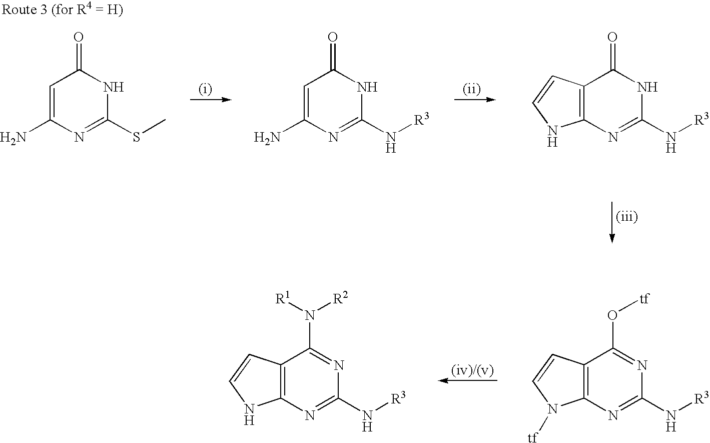
(i) R³NH₂, 90° C.; (ii) ClCH₂CHO, NaOAc, IPA/H₂O, 80; (iii) (CF₃SO₂)₂NPh, K₂CO₃, DMF, RT; (iv) HNR¹R², K₂CO₃, dioxane, microwave 80° C.; (v) 2N NaOH.

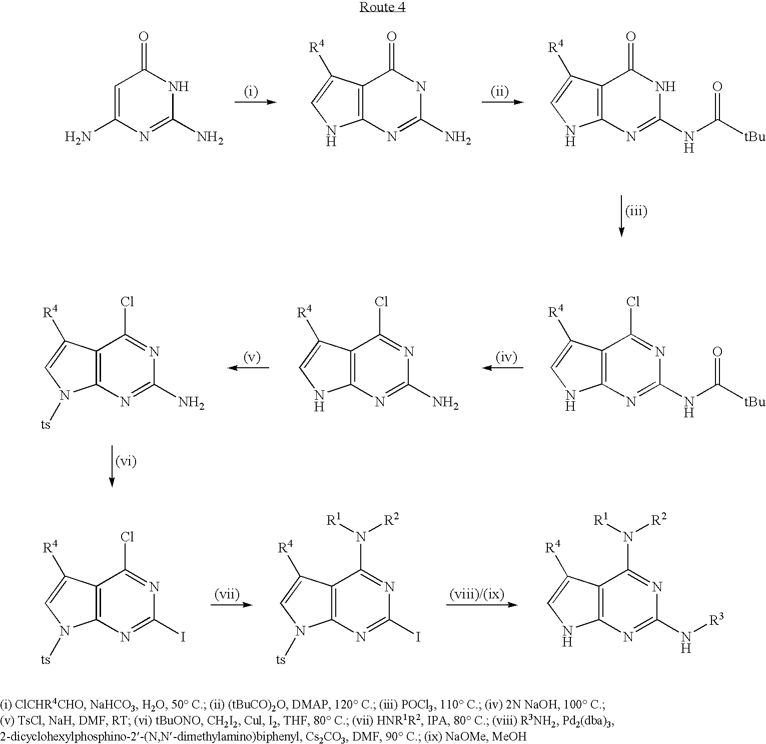

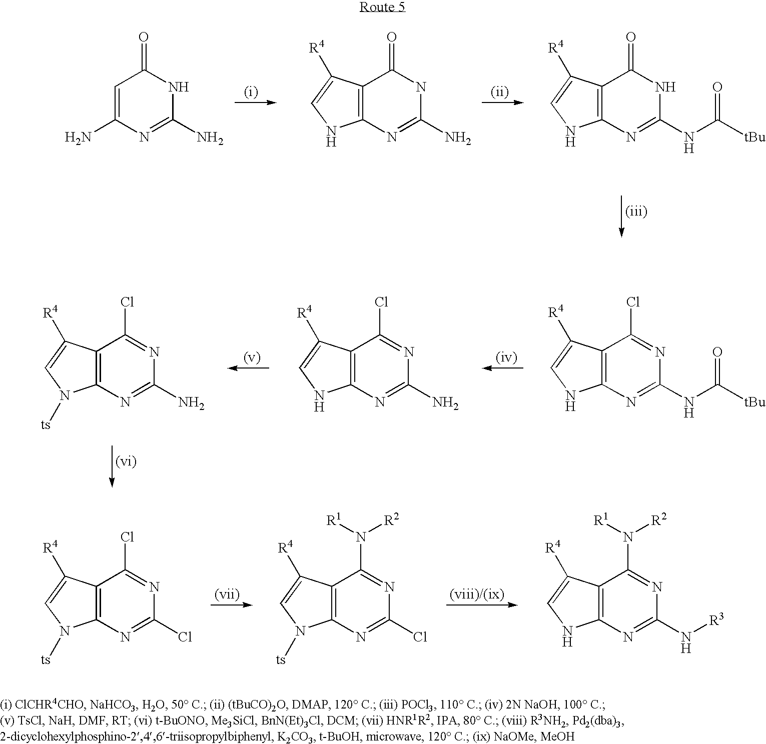

Accordingly, in a further aspect, the present invention provides a process for preparing a compound of formula (I) which process comprises:
(i) reacting a compound of formula (II): 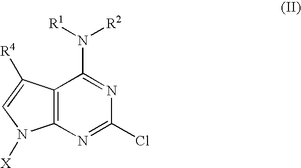
wherein X is H or a protecting group such as p-toluenesulphonyl, with an amine R³NH₂ and thereafter, if present, removing the protecting group;
(ii) when R⁴—H, reacting a compound of formula (III): 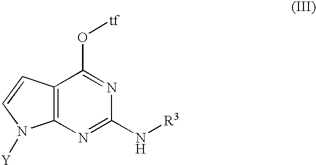
wherein Y is a protecting group such as triflate, with an amine HNR¹R² and thereafter removing the protecting group;
(iii) reacting a compound of formula (IV): 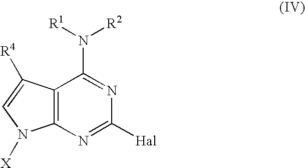
wherein Hal is Cl or I, with an amine R³NH₂ and thereafter removing the protecting group.

Certain embodiments of the present invention will now be illustrated by way of example only. The physical data given for the compounds exemplified is consistent with the assigned structure of those compounds.

### EXAMPLES

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:
g (grams);
l (liters);
μl (microliters);
M (molar);
MHz (megahertz);
mmol (millimoles);
min (minutes);
Rt (retention time);
TFA (trifluoroacetic acid);
THF (tetrahydrofuran);
DMSO (dimethylsulfoxide);
DCM (dichloromethane);
DMF (N,N-dimethylformamide);
DMAP (4-dimethylaminopyridine);
ATP (adenosine triphosphate);
DMEM (Dulbecco's modified Eagle medium);
HPLC (high pressure liquid chromatography);
TBAF (tetra-n-butylammonium fluoride);
TsCl (tosyl chloride);
HEPES (4-(2-hydroxyethyl)-1-piperazine ethane sulfonic acid);
EDTA (ethylenediaminetetraacetic acid);
TBTU (O-Benzotriazol-1-yl-N,N,N′,N′-tetramethyluronium tetrafluoroborate);
DIPEA (diisopropylethylamine);
Pd₂(dba)₃ (bis(dibenzylideneacetone)palladium);
LC/MS (liquid chromatography—mass spectrometry);
mg (milligrams);
ml (milliliters);
mM (millimolar);
h (hours);
IPA (isopropanol);
atm (atmosphere);
BSA (bovine serum albumin)
HRP (horseradish peroxidase);
MDAP (Mass directed autoprep/preparative mass directed HPLC);

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

¹H NMR spectra were recorded using a Bruker DPX 400 MHz, referenced to tetramethylsilane.

LC/MS was conducted on a Supelcosil LCABZ+PLUS column (3.3 cm×4.6 mm ID) eluting with 0.1% HCO₂H and 0.01M ammonium acetate in water (solvent A) and 0.05% HCO₂H 5% water in acetonitrile (solvent B), using the following elution gradient 0.0-7 min 0% B, 0.7-4.2 min 100% B, 4.2-5.3 min 0% B, 5.3-5.5 min 0% B at a flow rate of 3 ml/min. The mass spectra were recorded on a Fisons VG Platform spectrometer using electrospray positive and negative mode (ES+ve and ES−ve).

“Mass directed autoprep”/“preparative mass directed HPLC” was conducted on a system such as; a Waters FractionLynx system comprising of a Waters 600 pump with extended pump heads, Waters 2700 autosampler, Waters 996 diode array and Gilson 202 fraction collector on a 10 cm 2.54 cm ID ABZ+ column, eluting with either 0.1% formic acid or trifluoroacetic acid in water (solvent A) and 0.1% formic or trifluoroacetic acid in acetonitrile (solvent B) using the appropriate elution gradient. Mass spectra were recored on Micromass ZMD mass spectrometer using electrospray positive and negative mode, alternate scans. The software used was MassLynx 3.5 with OpenLynx and FractionLynx optio; or using equivalent alternative systems.

“Hydrophobic frits” refers to filtration tubes sold by Whatman. SPE (solid phase extraction) refers to the use of cartridges sold by International Sorbent Technology Ltd.

The Flashmaster II is an automated multi-user flash chromatography system, available from Argonaut Technologies Ltd, which utilises disposable, normal phase, SPE cartridges (2 g to 100 g). It provides quaternary on-line solvent mixing to enable gradient methods to be run. Samples are queued using the multi-functional open access software, which manages solvents, flow-rates, gradient profile and collection conditions. The system is equipped with a Knauer variable wavelength uv-detector and two Gilson FC204 fraction-collectors enabling automated peak cutting, collection and tracking.

Silica chromatography techniques include either automated (Flashmaster) techniques or manual chromatography on pre-packed cartridges (SPE) or manually-packed flash columns.

Microwave chemistry was typically performed in sealed vessels, irradiating with a suitable microwave reactor system, such as a Biotage Initiator™ Microwave Synthesiser.

When the name of a commercial supplier is given after the name of a compound or a reagent, for instance “compound X (Aldrich)” or “compound X/Aldrich”, this means that compound X is obtainable from a commercial supplier, such as the commercial supplier named.

Similarly, when a literature or a patent reference is given after the name of a compound, for instance compound Y (EP 0 123 456), this means that the preparation of the compound is described in the named reference.

The names of the Examples have been obtained using the compound naming programme “ACD Name Pro 6.02”.

### Example 1

### 4-({4-[(1-Methylethyl)amino]-1H-pyrrolo[2,3-d]pyrimidin-2-yl}amino)benzamide formate

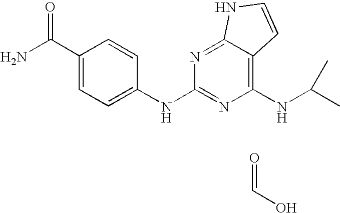

To a solution of 2-{[4-(aminocarbonyl)phenyl]amino}-7-[(trifluoromethyl)sulfonyl]-7H-pyrrolo[2,3-d]pyrimidin-4-yl trifluoromethanesulfonate (0.024 g) in dioxane (1.5 ml) was added potassium carbonate (15 mg) and isopropylamine (0.005 g). The suspension was heated in a sealed vial at 80° C. by microwave irradiation for 10 min. The mixture was treated with aqueous sodium hydroxide (2M, 0.75 ml) and stirred vigorously for 4 h. The mixture was treated with aqueous hydrochloric acid (2M, 0.75 ml) and applied to a SCX-2 cartridge (10 g, pre-conditioned with methanol). The cartridge was washed with methanol and eluted with 10% ammonia in methanol. The basic fractions were concentrated in vacuo and the residue purified by MDAP to give 4-({4-[(1-methylethyl)amino]-1H-pyrrolo[2,3-d]pyrimidin-2-yl}amino)benzamide formate (0.010 g) as a white solid. LC/MS: Rt 2.37 min, MH⁺ 311.

### Intermediate 1

### 2-{[4-(Aminocarbonyl)phenyl]amino}-7-[(trifluoromethyl)sulfonyl]-7H-pyrrolo[2,3-d]pyrimidin-4-yl trifluoromethanesulfonate

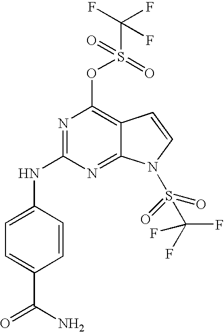

To a suspension of 4-[(4-oxo-4,7-dihydro-1H-pyrrolo[2,3-d]pyrimidin-2-yl)amino]benzamide (0.077 g) in DMF (3 ml) was added potassium carbonate (0.097 g) and N-phenyltrifluoromethanesuphonamide (0.25 g). The suspension was stirred at 20° C. for 1.5 h. A further amount of N-phenyltrifluoromethanesuphonamide (0.064 g) and potassium carbonate (0.024 g) was added to the mixture and stirred at 20° C. for 3.5 h. The mixture was partitioned between ethyl acetate (30 ml) and water (20 ml). The phases were separated and the organic phase washed with water (2×15 ml). The combined aqueous washings were extracted with ethyl acetate (20 ml) and the second ethyl acetate extract washed with water (10 ml). The combined organic extracts were dried (magnesium sulphate), filtered and the solvent removed in vacuo. The residue was adsorbed onto silica and purified by chromatography on a silica cartridge (20 g), eluting with an ethyl acetate/cyclohexane gradient (0-100%) over 30 min to give, after evaporation of the solvent from appropriate fractions, 2-{[4-(aminocarbonyl)phenyl]amino}-7-[(trifluoromethyl)sulfonyl]-7H-pyrrolo[2,3-d]pyrimidin-4-yl trifluoromethanesulfonate (0.050 g). LC/MS: Rt 3.50 min, MH⁺ 534.

### Intermediate 2

### 4-[(4-Oxo-4,7-dihydro-1H-pyrrolo[2,3-d]pyrimidin-2-yl)amino]benzamide

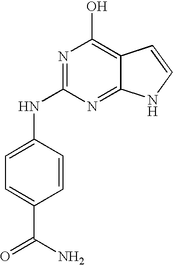

To a suspension of 4-[(4-amino-6-oxo-1,6-dihydro-2-pyrimidinyl)amino]benzamide (0.325 g) in IPA (3 ml) and water (1 ml) was added sodium acetate (0.240 g). To the mixture was added chloroacetaldehyde (0.22 ml, 50% in water). The suspension was heated to 80° C. for 20 min. The mixture, at room temperature, was diluted with water (30 ml) and the resulting suspension stirred for 15 min. The suspension was filtered and the residue washed with water (10 ml). The crude was further purified by chromatography on a silica cartridge (50 g), eluting with a methanol/DCM gradient (0-30%)+1% triethylamine to give, after evaporation of the solvents from appropriate fractions, 4-[(4-oxo-4,7-dihydro-1H-pyrrolo[2,3-d]pyrimidin-2-yl)amino]benzamide (0.132 g) as a white solid. LC/MS: Rt 2.1 min, MH⁺ 270.

### Intermediate 3

### 4-[(4-Amino-6-oxo-1,6-dihydro-2-pyrimidinyl)amino]benzamide

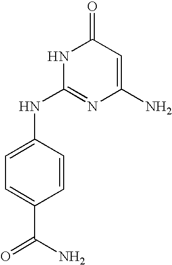

A mixture of 6-amino-2-(methylthio)-4(1H)-pyrimidinone (1.023 g, Salor) and 4-aminobenzamide (1.0 g, Acros) was shaken at room temperature and then stirred at 190° C. for 26 h. The residue was adsorbed onto silica using DCM/methanol (1:1, 100 ml). The crude product was purified by chromatography on a silica cartridge (100 g), eluting with a methanol/DCM gradient (0-25%) and then with 50% methanol/DCM with 1% triethylamine. Evaporation of the solvent from appropriate fractions gave 4-[(4-amino-6-oxo-1,6-dihydro-2-pyrimidinyl)amino]benzamide (0.340 g) as a yellow solid. LC/MS: Rt 1.8 min, MH⁺ 246.

### Intermediate 4

### 2-Iodo-N-(1-methylethyl)-7-[(4-methyl phenyl)sulfonyl]-7H-pyrrolo[2,3-d]pyrimidin-4-amine

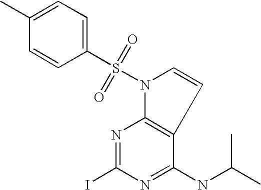

4-Chloro-2-iodo-7-[(4-methylphenyl)sulfonyl]-7H-pyrrolo[2,3-d]pyrimidine (1.3 g) was suspended in ethanol (20 ml) and treated with isopropylamine (360 mg, Aldrich) and DIPEA (10 mmol) and the mixture was heated at 80° C. for 3 h. The reaction was reduced to dryness and the residue purified by chromatography on a silica cartridge, eluting with an ethyl acetate/DCM gradient (0-100%). Combination of the appropriate fractions and evaporation of the solvents gave the title compound (950 mg). LC/MS; Rt 3.88 min, MH⁺ 456.9.

### Intermediate 5

### 2-Iodo-7-[(4-methyl phenyl)sulfonyl]-N-(2,2,2-trifluoroethyl)-7H-pyrrolo[2,3-d]pyrimidin-4-amine

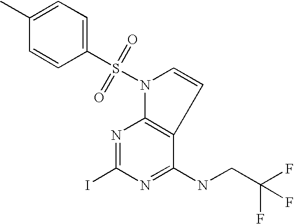

4-Chloro-2-iodo-7-[(4-methylphenyl)sulfonyl]-7H-pyrrolo[2,3-d]pyrimidine (1.3 g) was suspended in ethanol (20 ml) and treated with 2,2,2-trifluorethylamine (600 mg, Aldrich) and DIPEA (10 mmol) and the mixture was heated at 80° C. for 6 h. 2,2,2-Trifluorethylamine (2 ml) and DIPEA (2 ml) were added and heating continued at 90° C. for 18 h. The reaction was reduced to dryness and the residue purified by chromatography on a silica cartridge, eluting with an ethyl acetate/DCM gradient (0-100%). Combination of the appropriate fractions and evaporation of the solvents gave the title compound (1.21 g). LC/MS; Rt 3.80 min, MH⁺ 496.9

Method 1:

4-Chloro-2-iodo-7-[(4-methylphenyl)sulfonyl]-7H-pyrrolo[2,3-d]pyrimidine (310 mg) was suspended in ethanol and treated with amine (2 mmol) and DIPEA (3 mmol) and the mixture was heated at 80° C. for 3 h. The reaction was reduced to dryness and the residue purified by chromatography on a silica cartridge, eluting with an ethyl acetate/DCM gradient (0-100%). Combination of the appropriate fractions and evaporation of the solvents gave the desired product

The following compounds were prepared using Method 1:

| Inter- | | | Amine/ | LC/MS | LC/MS |
|---|---|---|---|---|---|
| mediate | Structure | Name | source | Rt (min) | MH⁺ |
| 6 | 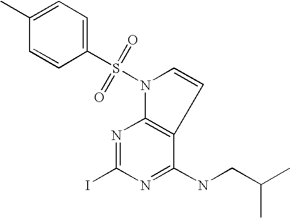 | 2-iodo-7-[(4-methylphenyl)sulfonyl]- N-(2-methylpropyl)-7H-pyrrolo [2,3-d]pyrimidin-4-amine | Isobutylamine/ Aldrich | 4.09 | 470.83 |
| 7 | 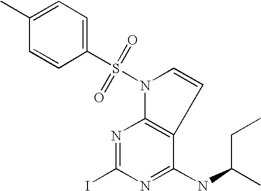 | 2-iodo-7-[(4-methylphenyl)sulfonyl]- N-[(1R)-1-methylpropyl]-7H- pyrrolo[2,3-d]pyrimidin-4-amine | (R)-sec Butylamine/ Aldrich | 4.04 | 470.82 |

### Intermediate 8

### N-Ethyl-2-iodo-7-[(4-methyl phenyl)sulfonyl]-7H-pyrrolo[2,3-d]pyrimidin-4-amine

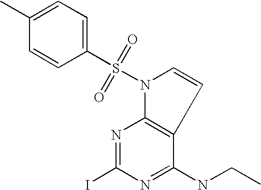

4-Chloro-2-iodo-7-[(4-methylphenyl)sulfonyl]-7H-pyrrolo[2,3-d]pyrimidine (300 mg) was suspended in ethanol (5 ml) and treated with ethylamine (1 ml, Aldrich) and DIPEA (1 ml) and the mixture was heated at 80° C. for 2 h. The reaction was reduced to dryness and the residue purified by chromatography on a silica cartridge (20 g), eluting with an ethyl acetate/cyclohexane gradient (0-100%). Combination of the appropriate fractions and evaporation of the solvents gave the title compound. LC/MS; Rt 3.82 min, MH⁺ 442.78.

Method 2:

Pyrrolo[2,3-d]pyrimidin-4-amine reagent, for example, Pyrrolo[2,3-d]pyrimidin-4-amine (0.1 mmol, 43 mg), 4-amino-N-methylbenzamide (29.8 mg, Asinex), cesium carbonate (96 mg), bis(dibenzylideneacetone)palladium (6 mg, Acros) and 2-dicyclohexylphosphino-2′-(N,N-dimethylamino)biphenyl (6 mg, Acros) were combined in DMF (2.0 ml). The reaction mixture was heated at 80° C. for 3 h. The reaction mixture was allowed to cool, filtered through Celite, the Celite washed with DMF and the combined filtrate and washings evaporated to dryness. The residue was heated with sodium methoxide solution (2N, 0.5 ml) at 80° C. for 2 h and allowed to cool to room temperature. The solution was evaporated to dryness, the residue dissolved in DMSO and purified by MDAP. The fractions containing product were evaporated to dryness to give the desired compound.

| The | | | | | |
|---|---|---|---|---|---|
| following | | | | | |
| were | | | | | |
| prepared | | | | | |
| using | | | | | |
| Method | | | Pyrrolo[2,3-d]pyrimidin- | LC/MS | LC/MS |
| 2: Example | Structure | Name | 4-amine Reagent | Rt (min) | MH⁺ |
| 2 | 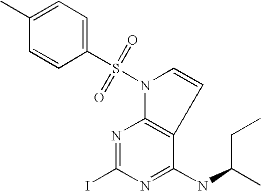 | N-methyl-4-[(4-{[(1R)- 1-methylpropyl]amino}- 1H-pyrrolo[2,3-d]pyrimidin-2-yl)amino]benzamide trifluoroacetate | 2-iodo-7-[(4-methylphenyl) sulfonyl]-N-[(1R)- 1-methylpropyl]-7H- pyrrolo[2,3-d]pyrimidin-4-amine | 2.40 | 339 |
| 3 | 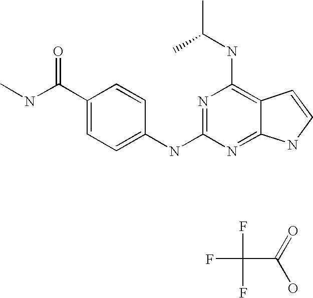 | 4-{[4-(ethylamino)-1H- pyrrolo[2,3-d]pyrimidin- 2-yl]amino}-N-methyl benzamide trifluoroacetate | N-ethyl-2-iodo-7-[(4- methylphenyl)sulfonyl]- 7H-pyrrolo[2,3-d]pyrimidin-4-amine | 2.17 | 311 |
| 4 | 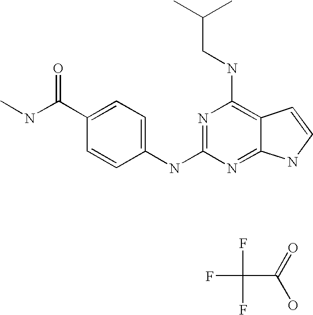 | N-methyl-4-({4-[(2- methylpropyl)amino]-1H- pyrrolo[2,3-d]pyrimidin- 2-yl}amino)benzamide trifluoroacetate | 2-iodo-7-[(4-methylphenyl) sulfonyl]-N-(2- methylpropyl]-7H- pyrrolo[2,3-d]pyrimidin-4-amine | 2.45 | 339 |

Method 3:

2-{[4-(Aminocarbonyl)phenyl]amino}-7-[(trifluoromethyl)sulfonyl]-7H-pyrrolo[2,3-d]pyrimidin-4-yl trifluoromethanesulfonate (853 mg) was suspended in IPA (16 ml) and an aliquot (1 ml) of this mixture treated with a solution of the amine (0.15 mmol) in IPA (1 ml) and DIPEA (17 μl). The reaction was stirred at 80° C. under reflux conditions for overnight. The reaction was concentrated and the residue dissolved in dioxane (1 ml) and sodium hydroxide (2M, 1 ml) the resulting biphasic mixture was stirred vigorously at room temperature for ˜72 h. The reaction was neutralised with hydrochloric acid (2N), and extracted with ethyl acetate (2 ml). The organic phase was concentrated and the residue purified by MDAP. The fractions containing product were evaporated to dryness to give the desired compound.

The following compounds were prepared using Method 3:

| | | | Amine | LC/MS | |
|---|---|---|---|---|---|
| | | | Reagent/ | Rt | LC/MS |
| Example | Structure | Name | Source | (min) | MH⁺ |
| 5 | 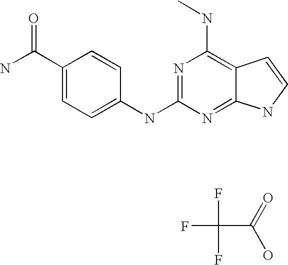 | 4-{[4-(methylamino)-1H- pyrrolo[2,3-d]pyrimidin-2-yl]amino}benzamide trifluoroacetate | Methylamine/ Acros | 2.03 | 283 |
| 6 | 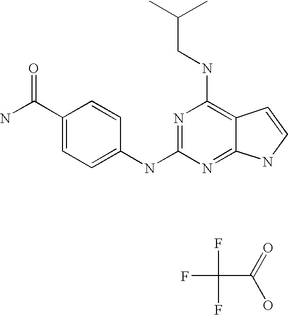 | 4-({4-[(2-methylpropyl) amino]-1H-pyrrolo[2,3-d]pyrimidin-2-yl}amino) benzamide trifluoroacetate | (2-methylpropyl) amine/ Aldrich | 2.39 | 325 |
| 7 | 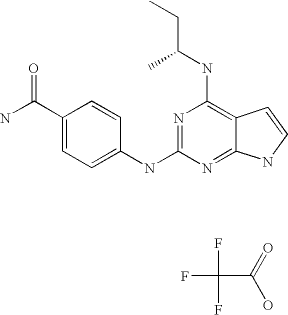 | 4-[(4-{[(1R)-1-methylpropyl]amino}-1H-pyrrolo[2,3-d]pyrimidin-2-yl)amino]benzamide trifluoroacetate | [(1S)-1- methylpropyl]amine/ Acros | 2.34 | 325 |
| 8 | 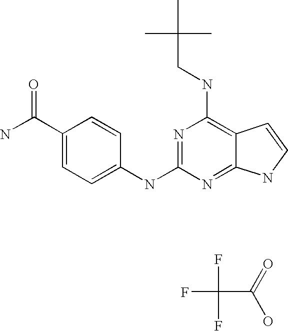 | 4-({4-[(2,2-dimethylpropyl) amino]-1H-pyrrolo[2,3-d]pyrimidin-2-yl}amino) benzmide trifluoroacetate | (2,2- dimethylpropyl) amine/ Fluorochem | 2.51 | 339 |

Method 4:

2-{[4-(Aminocarbonyl)phenyl]amino}-7-[(trifluoromethyl)sulfonyl]-7H-pyrrolo[2,3-d]pyrimidin-4-yl trifluoromethanesulfonate (1190 mg, 60% purity) was suspended in IPA (17 ml). An aliquot (1 ml) of this mixture was treated with a solution of the amine (0.15 mmol) in IPA (1 ml) and DIPEA (17 μl). The reaction was stirred at 80° C. under reflux conditions for overnight. The reaction was concentrated under a stream of nitrogen and the residue dissolved in dioxane (1 ml) and sodium hydroxide (2M, 1 ml) the resulting biphasic mixture was stirred vigorously at 25° C. for ˜72 h. The dioxane phase was isolated and concentrated. The residue was purified by MDAP. Appropriate fractions were evaporated to dryness to give the desired product.

The following compounds were prepared using Method 4:

| | | | Amine | LC/MS | |
|---|---|---|---|---|---|
| | | | Reagent/ | Rt | LC/MS |
| Example | Structure | Name | Source | (min) | MH⁺ |
| 9 | 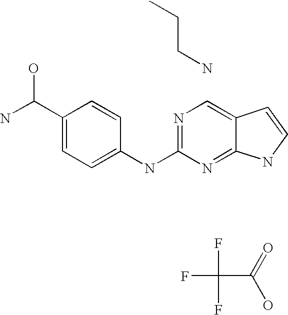 | 4-{[4-(propylamino)-1H- pyrrolo[2,3-d]pyrimidin- 2-yl]amino}benzamide trifluoroacetate | 1-propylamine/ Aldrich | 2.24 | 311 |
| 10 | 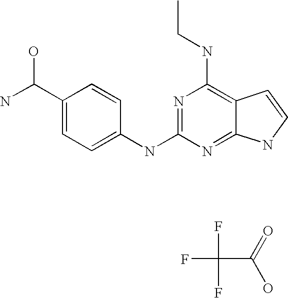 | 4-{[4-(ethylamino)-1H- pyrrolo[2,3-d]pyrimidin- 2-yl]amino}benzamide trifluoroacetate | ethylamine Aldrich | 2.12 | 297 |
| 11 | 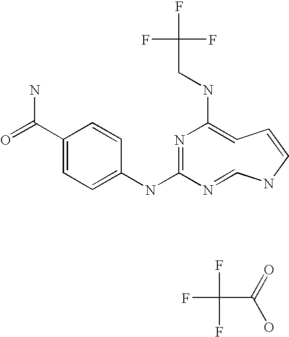 | 4-({4-[(2,2,2-trifluoroethyl) amino]-1H-pyrrolo [2,3-d]pyrimidin-2-yl}amino)benzamide trifluoroacetate | (2,2,2-trifluoro ethyl)amine/ Aldrich | 2.51 | 351 |
| 9 | 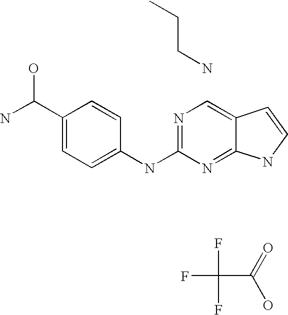 | 4-{[4-(propylamino)-1H- pyrrolo[2,3-d]pyrimidin- 2-yl]amino}benzamide trifluoroacetate | 1-propylamine/ Aldrich | 2.24 | 311 |
| 10 | 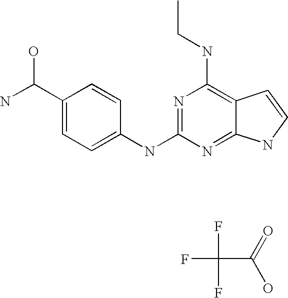 | 4-{[4-(ethylamino)-1H- pyrrolo[2,3-d]pyrimidin- 2-yl]amino}benzamide trifluoroacetate | ethylamine Aldrich | 2.12 | 297 |
| 11 | 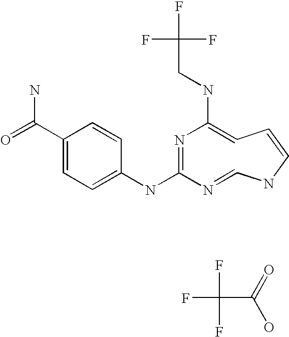 | 4-({4-[(2,2,2-trifluoroethyl) amino]-1H-pyrrolo [2,3-d]pyrimidin-2-yl}amino)benzamide trifluoroacetate | (2,2,2-trifluoro ethyl)amine/ Aldrich | 2.51 | 351 |

### Example 12

### 4-({4-[(2,2-Difluoropropyl)amino]-1H-pyrrolo[2,3-d]pyrimidin-2-yl}amino)benzamide trifluoroacetate

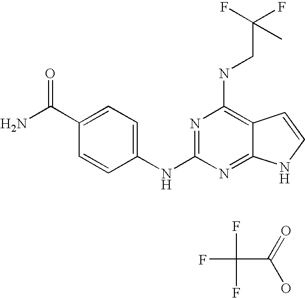

2-{[4-(Aminocarbonyl)phenyl]amino}-7-[(trifluoromethyl)sulfonyl]-7H-pyrrolo[2,3-d]pyrimidin-4-yl trifluoromethanesulfonate (312 mg) was suspended in IPA (17 ml). An aliquot (1 ml) of this mixture was treated with a solution of the (2,2-difluoropropyl)amine (14.3 mg, Oakwood Products) in IPA (1 ml) and DIPEA (17 μl). The reaction was stirred at 80° C. under reflux conditions for 18 h. The reaction was concentrated and the residue dissolved in dioxane (1 ml) and sodium hydroxide (2M, 1 ml) the resulting biphasic mixture was stirred vigorously at 25° C. for ˜90 h. The dioxane phase was isolated and concentrated. The residue was purified by MDAP. Appropriate fractions were evaporated to dryness to give the title compound. LC/MS; Rt 2.43 min, MH⁺ 347.

### Example 13

### 4-({4-[(3-Methylbutyl)amino]-1H-pyrrolo[2,3-d]pyrimidin-2-yl}amino)benzamide trifluoroacetate

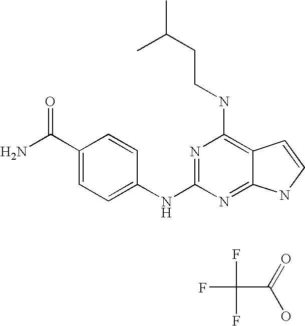

2-{[4-(Aminocarbonyl)phenyl]amino}-7-[(trifluoromethyl)sulfonyl]-7H-pyrrolo[2,3-d]pyrimidin-4-yl trifluoromethanesulfonate (2.7 g, impure ˜4.2 mmol) was suspended in IPA (42 ml). An aliquot (1 ml) of this mixture was treated with (3-methylbutyl)amine (13.1 mg, Aldrich) in IPA (1 ml) and DIPEA (17 μl). The reaction was stirred at 80° C. under reflux conditions for ˜72 h. The reaction was concentrated (vacuum centrifuge), the residue dissolved in methanol (1.5 ml) and treated with sodium methoxide in methanol (0.5M, 0.5 ml) the resulting solution was stirred at 80° C. overnight. The reaction was concentrated (vacuum centrifuge) and the residue purified by MDAP. The fractions containing product were evaporated to dryness to give the title compound (13.8 mg) (Purification method 1). LC/MS; Rt 2.63 min, MH⁺ 339.

The following compounds were prepared in a similar manner, and purified using either the purification method above (Purification method 1) or Purification method 2 (below).

Purification method 2

After deprotection with sodium methoxide, conversion to the deprotected species was incomplete. The reaction was concentrated and the residue redissolved in dioxane (1 ml) and sodium hydroxide (2M, 1 ml). The reaction was stirred vigorously for 16 h. The dioxane phase was isolated, concentrated and the residue purified by MDAP. The appropriate fractions were evaporated to dryness to give the desired product.

| | | | Amine | | LC/MS | |
|---|---|---|---|---|---|---|
| | | | Reagent/ | Purification | Rt | LC/MS |
| Example | Structure | Name | Source | method | (min) | MH⁺ |
| 14 | 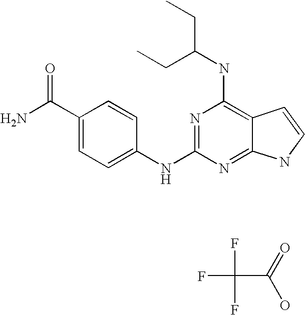 | 4-({4-[(1-ethylpropyl) amino]-1H-pyrrolo[2, 3-d]pyrimidin-2-yl}amino)benzamide trifluoroacetate | (1-ethylpropyl) amine/ Aldrich | 1 | 2.52 | 339 |
| 15 | 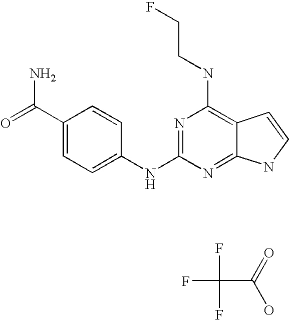 | 4-({4-[(2-fluoroethyl) amino]-1H-pyrrolo[2, 3-d]pyrimidin-2-yl}amino)benzamide trifluoroacetate | (2-fluoroethyl) amine hydrochloride/ Aldrich | 2 | 2.21 | 315 |
| 16 | 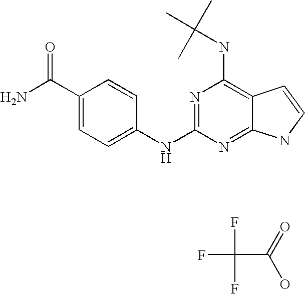 | 4-({4-[(1,1-dimethylethyl) amino]-1H-pyrrolo [2,3-d]pyrimidin-2-yl}amino)benzamide trifluoroacetate | (1,1-dimethylethyl) amine/ Aldrich | 1 | 2.48 | 325 |
| 17 | 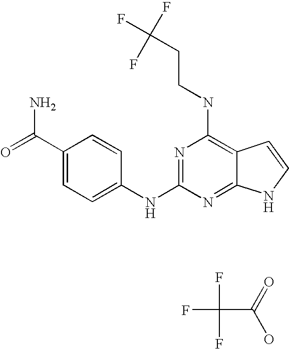 | 4-({4-[(3,3,3-trifluoro propyl)amino]-1H-pyrrolo [2,3-d]pyrimidin-2- yl}amino)benzamide trifluoroacetate | (3,3,3-trifluoro propyl)amine hydrochloride/ Apollo | 2 | 2.53 | 365 |
| 18 | 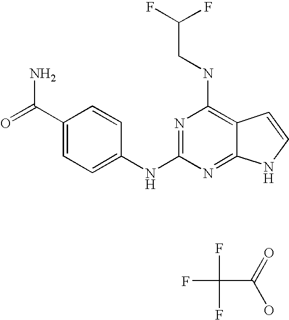 | 4-({4-[(2,2-difluoroethyl) amino]-1H-pyrrolo [2,3-d]pyrimidin-2-yl}amino)benzamide trifluoroacetate | (2,2-difluoroethyl) amine/ Apollo | 1 | 2.39 | 333 |

### Example 19

### 4-({4-[(1-Methylethyl)amino]-1H-pyrrolo[2,3-d]pyrimidin-2-yl}amino)benzamide trifluoroacetate

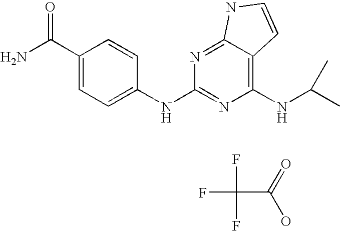

A mixture of 2-iodo-N-(1-methylethyl)-7-[(4-methyl phenyl)sulfonyl]-7H-pyrrolo[2,3-d]pyrimidin-4-amine (45.8 mg), 4-aminobenzamide (20.4 mg, Aldrich), cesium carbonate (97.5 mg), 2-dicyclohexylphosphino-2′-(N,N-dimethylamino)biphenyl (5.8 mg) and bis(dibenzylideneacetone) palladium (5.8 mg) was suspended in DMF (2 ml) and the reaction was stirred at 80° C. under nitrogen for 4 h. The reaction was filtered through Celite and the filtrate concentrated. The resulting gum was treated with 4-aminobenzamide (20.4 mg), cesium carbonate (130 mg), 2-dicyclohexylphosphino-2′-(N,N-dimethylamino)biphenyl (5.8 mg) and bis(dibenzylideneacetone)palladium (5.8 mg) in DMF (2 ml) and the reaction was stirred at 80° C. under nitrogen for 2 h. The reaction was filtered through Celite and concentrated. The residue was dissolved in methanol (1 ml), treated with sodium methoxide in methanol (0.5M, 1 ml) and stirred at 60° C. overnight. The reaction was concentrated and the residue purified by MDAP. The fractions containing product were evaporated to dryness to give the title compound (6.0 mg). LC/MS; Rt 2.22 min, MH⁺ 311.

Method 5:

2-Iodo-7-[(4-methyl phenyl)sulfonyl]-N-(2,2,2-trifluoroethyl)-7H-pyrrolo[2,3-d]pyrimidin-4-amine (992 mg) was suspended in DMF (20 ml). An aliquot (1 ml) of this mixture was treated with a solution of the aniline (0.2 mmol) in DMF (1 ml), cesium carbonate (97.5 mg), 2-dicyclohexylphosphino-2′-(N,N-dimethylamino)biphenyl (5.8 mg) and bis(dibenzylideneacetone) palladium (5.8 mg). The reaction was stirred at 80° C. under nitrogen for 3 h. The reaction was filtered through Celite, concentrated (vacuum centrifuge) and the residue dissolved in methanol (1 ml), treated with sodium methoxide in methanol (0.5M, 500 μl) and stirred at 60° C. overnight. The reaction was concentrated and purified using MDAP. The appropriate fractions were reduced to dryness to give the title compound.

The following were prepared using Method 5:

| | | | Aniline | | LC/MS | |
|---|---|---|---|---|---|---|
| | | | Reagent/ | Purifi- | Rt | LC/MS |
| Example | Structure | Name | Source | cation | (min) | MH⁺ |
| 20 | 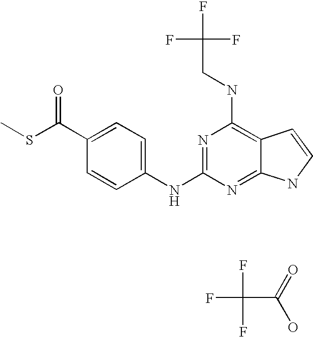 | N-methyl-4-({4-[(2,2,2- trifluoroethyl)amino]-1H- pyrrolo[2,3-d]pyrimidin- 2-yl}amino)benzamide trifluoroacetate | 4-amino-N-methyl benzamide/Park research | 3 | 2.59 | 365 |
| 21 | 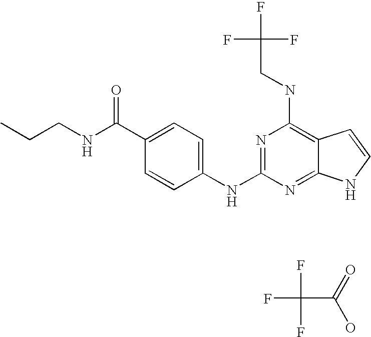 | N-propyl-4-({4-[(2,2,2- trifluoroethyl)amino]-1H- pyrrolo[2,3-d]pyrimidin- 2-yl}amino)benzamide trifluoroacetate | 4-amino-N-propyl benzamide/Park research | 4 | 2.87 | 393 |

Purification:

(3) MDAP

(4) Purified by 2 sequential MDAPs

### Example 22

### N-Propyl-4-({4-[(2,2,2-trifluoroethyl)amino]-1H-pyrrolo[2,3-d]pyrimidin-2-yl}amino)benzamide

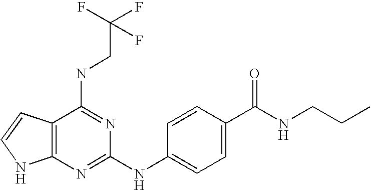

4-({7-[(4-Methylphenyl)sulfonyl]-4-[(2,2,2-trifluoroethyl)amino]-7H-pyrrolo[2,3-d]pyrimidin-2-yl}amino)-N-propylbenzamide (550 mg) and potassium carbonate (414 mg) in methanol/water (4:1, 12.5 ml) was heated at reflux for 5 h. The cooled reaction was diluted with water and the precipitate isolated by filtration. The solid was washed with ether to leave the title compound as a white solid (315 mg). LC/MS; Rt 3.10 min, MH⁺ 393.

### Intermediate 9

### 4-({7-[(4-Methyl phenyl)sulfonyl]-4-[(2,2,2-trifluoroethyl)amino]-7H-pyrrolo[2,3-d]pyrimidin-2-yl}amino)-N-propylbenzamide

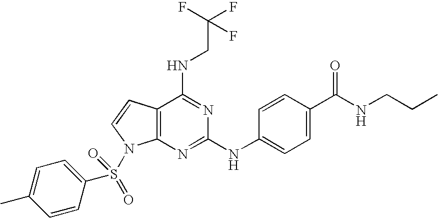

A mixture of 2-chloro-7-[(4-methylphenyl)sulfonyl]-N-(2,2,2-trifluoroethyl)-7H-pyrrolo[2,3-d]pyrimidin-4-amine (500 mg), 4-amino-N-propylbenzamide (267 mg, Buttpark Screening Library), tris(dibenzylideneacetone)dipalladium (68 mg), 2-dicyclohexylphosphino-2′,4′,6′-triisopropylbiphenyl (30 mg) and potassium carbonate (222 mg) in t-butanol (10 ml) was heated at reflux under nitrogen overnight. The cooled reaction was partitioned between ethyl acetate and water and the organic phase washed with water and brine. The organic phase was dried (hydrophobic frit) and reduced to dryness in vacuo. The residue was purified by chromatography on a silica cartridge (50 g) eluting with an ethyl acetate/cyclohexane gradient (1:15 to 7:1). The solvents were evaporated from the product fractions to leave the title compound (556 mg). LC/MS; Rt 3.5 min, MH⁺ 547.

### Intermediate 10

### 2-Chloro-N-(1-methylethyl)-7-[(4-methylphenyl)sulfonyl]-7H-pyrrolo[2,3-d]pyrimidin-4-amine

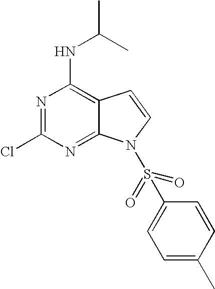

To a suspension of 2,4-dichloro-7-[(4-methylphenyl)sulfonyl]-7H-pyrrolo[2,3-d]pyrimidine (70 g) in IPA (900 ml) was added isopropylamine (70 ml). The mixture was heated at 100° C. for 30 min then concentrated in vacuo. The residue was partitioned between water (1.51) and ethyl acetate (300 ml). The layers were separated and the aqueous phase was further extracted with ethyl acetate (2×300 ml). The combined organic extracts were dried over sodium sulphate and evaporated in vacuo. The residue was evaporated from ether to give the title compound as a gold coloured foam (72.2 g). NMR [CDCl₃]; δH 8.10, (2H, d), 7.43, (1H, d), 7.33, (2H, d), 6.39, (1H, d), 4.97, (1H, br s), 4.37, (1H, br m), 2.41, (3H, s), 1.27, (6H, d). LC/MS; Rt 3.59 min, MH⁺ 365, 367.

### Intermediate 11

### 2,4-Dichloro-7-[(4-methylphenyl)sulfonyl]-7H-pyrrolo[2,3-d]pyrimidine

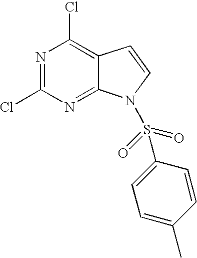

To a solution of 4-chloro-7-[(4-methylphenyl)sulfonyl]-7H-pyrrolo[2,3-d]pyrimidin-2-amine (86.8 g), chlorotrimethylsilane (570 ml) and benzyl triethylammonium chloride (127.2 g) in DCM (1.11), under a nitrogen atmosphere, was added tert-butyl nitrite (52 ml) dropwise over 20 min. After stirring for 15 min the mixture was cooled to ˜20° C. and treated cautiously with water (1.51) whilst cooling the mixture in an ice bath. The layers were separated and the aqueous phase was further extracted with DCM (2×500 ml). The combined organic extracts were dried (sodium sulphate) and evaporated in vacuo. The residue was triturated with ether to give the title compound as a pale yellow solid (70.6 g). NMR; [CDCl₃] δH 8.12, (2H, d), 7.76, (1H, d), 7.37, (2H, d), 6.68, (1H, d), 2.44, (3H, s). LC/MS; Rt 3.54 min, MH⁺ 342, 344, 346.

### Example 23

### N-Methyl-4-({4-[(2,2,2-trifluoroethyl)amino]-1H-pyrrolo[2,3-d]pyrimidin-2-yl}amino)benzamide

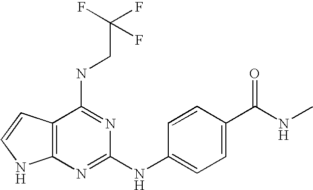

N-Methyl-4-({7-[(4-methylphenyl)sulfonyl]-4-[(2,2,2-trifluoroethyl)amino]-7H-pyrrolo[2,3-d]pyrimidin-2-yl}amino)benzamide (385 mg) and sodium methoxide in methanol (0.5M, 5 ml) were heated at 80° C. for 1.5 h. The reaction was left to cool to room temperature overnight, the methanol evaporated in vacuo, the residue triturated with water and filtered. The residual solid was adsorbed onto silica, applied to a silica cartridge (20 g) and the cartridge eluted with an ethyl acetate/cyclohexane gradient (30-100%). The product fraction was reduced to dryness under vacuum, and the residue triturated with ether/ethyl acetate to give the title compound as a white solid (115 mg). LC/MS; Rt 2.65 min, MH⁺ 365.

### Intermediate 12

### N-Methyl-4-({7-[(4-methyl phenyl)sulfonyl]-4-[(2,2,2-trifluoroethyl)amino]-7H-pyrrolo[2,3-d]pyrimidin-2-yl}amino)benzamide

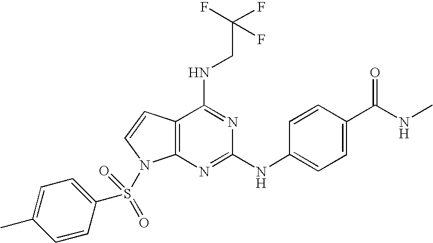

A mixture of 2-chloro-7-[(4-methylphenyl)sulfonyl]-N-(2,2,2-trifluoroethyl)-7H-pyrrolo[2,3-d]pyrimidin-4-amine (404 mg), 4-amino-N-methylbenzamide (180 mg), tris(dibenzylideneacetone)dipalladium (0) (91.6 mg), 2-dicyclohexylphosphino-2′,4′,6′-triisopropylbiphenyl (47.3 mg) and potassium carbonate (193 mg) in t-butanol (18 ml) was degassed and then heated at 80° C. under nitrogen overnight. The cooled reaction was diluted with ethyl acetate, applied to a SCX-2 SPE (50 g), the column washed with ethyl acetate and methanol and the product eluted with methanol/0.880 ammonia. The solvents were evaporated to give the title compound as a beige foam (385 mg). LC/MS; Rt 3.52 min, MH⁺ 519.

### Intermediate 13

### 4-Chloro-7-[(4-methylphenyl)sulfonyl]-7H-pyrrolo[2,3-d]pyrimidin-2-amine

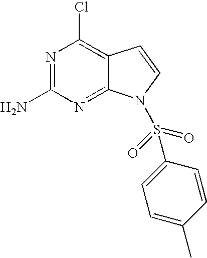

Sodium hydride (60% dispersion in oil, 2.2 g) was added to a stirred cooled (ice-bath) solution of 4-chloro-1H-pyrrolo[2,3-d]pyrimidin-2-amine (8.0 g, WO2004024082) in DMF (120 ml) under nitrogen. After 15 min, a solution of 4-toluenesulphonyl chloride (11 g) in DMF (50 ml) was added over 10 min. The mixture was stirred for 25 min and poured into a 10% ammonium chloride solution (800 ml) and extracted into ethyl acetate (3×200 ml). The combined extracts were washed with water (3×200 ml), dried (sodium sulphate) and evaporated in vacuo to give the title compound as a yellow solid (15 g). LC/MS; Rt 3.34 min, MH⁺ 325.

### Intermediate 14

### 4-Chloro-2-iodo-7-[(4-methylphenyl)sulfonyl]-7H-pyrrolo[2,3-d]pyrimidine

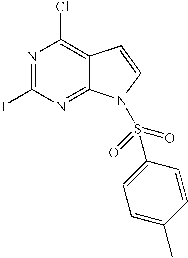

tert-Butyl nitrite (23 ml) was added to a stirred mixture of 4-chloro-7-[(4-methylphenyl)sulfonyl]-7H-pyrrolo[2,3-d]pyrimidin-2-amine (15 g), cuprous iodide (10.6 g), iodine (13.7 g) and diiodomethane (44 ml) in THF (250 ml) at room temperature. The mixture was then heated to 80° C. over 20 min and kept at this temperature for 45 min. The cooled reaction mixture was poured into an aqueous solution of sodium sulphite (1000 ml) and extracted into ethyl acetate (3×300 ml). The combined extracts were washed with water (2×300 ml) dried (sodium sulphate) and the solvent evaporated. The residue was purified by flash chromatography on silica (800 g), eluting with cyclohexane/ether (3:1). The appropriate fractions were evaporated to give the title compound as an off-white solid (8.5 g). LC/MS; Rt 3.74 min, MH⁺ 435.

### Intermediate 15

### 2-Chloro-7-[(4-methyl phenyl)sulfonyl]-N-(2,2,2-trifluoroethyl)-7H-pyrrolo[2,3-d]pyrimidin-4-amine

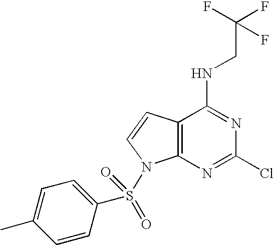

A mixture of 2,4-dichloro-7-[(4-methylphenyl)sulfonyl]-7H-pyrrolo[2,3-d]pyrimidine (4.0 g), 2,2,2-trifluoroethylamine (1.49 g, Aldrich), DIPEA (3.23 ml) and ethanol (100 ml) was heated at 95° C. under nitrogen overnight. The reaction mixture was concentrated, the residue dissolved in ethyl acetate (500 ml) and washed with water (5×300 ml), and the organic phase concentrated. The residue was dissolved in ethanol (100 ml), 2,2,2-trifluoroethylamine (1.49 g, Aldrich), DIPEA (3.23 ml) added and the mixture heated at 95° C. under nitrogen overnight. The mixture was concentrated, the residue dissolved in ethyl acetate (450 ml) and washed with water (5×200 ml). The organic phase was dried (hydrophobic frit) and concentrated to give the title compound (4.43 g). LC/MS; Rt 3.64 min, MH⁺ 405.

### Example 24

### N-Propyl-4-({4-[(2,2,2-trifluoroethyl)amino]-1H-pyrrolo[2,3-d]pyrimidin-2-yl}amino)benzamide

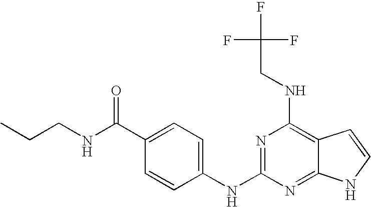

To 4-({7-[(4-methylphenyl)sulfonyl]-4-[(2,2,2-trifluoroethyl)amino]-7H-pyrrolo[2,3-d]pyrimidin-2-yl}amino)-N-propylbenzamide (101 g) was added methanol (1500 ml) followed by water (500 ml) and solid potassium carbonate (76.5 g). The initial solution rapidly became cloudy as it was heated to reflux. After 5 h at reflux the reaction was cooled and filtered. The isolated white solid washed with water (˜1.51) and sucked dry on the filter. This solid was suspended in water containing 5% methanol by volume (500 ml), another 500 ml of methanol/water was added and the mixture stirred well for 1 h, filtered under vacuum and washed with methanol/water (250 ml). The solid was sucked dry and then further dried under high vacuum at 40° C., to give the desired product as a white solid (60.3 g). LC/MS; Rt 2.90 min, MH⁺ 393. NMR; [D₆-DMSO] δH 11.22, (1H, s), 9.10, (1H, s), 8.19, (1H, t), 7.93-7.86, (3H, m), 7.73, (2H, d), 6.89, (1H, m), 6.51, (1H, m), 4.38, (2H, m), 3.20, (2H, q), 1.53, (2H, m), 0.89, (3H, t).

### Intermediate 16

### 4-({7-[(4-Methyl phenyl)sulfonyl]-4-[(2,2,2-trifluoroethyl)amino]-7H-pyrrolo[2,3-d]pyrimidin-2-yl}amino)-N-propylbenzamide

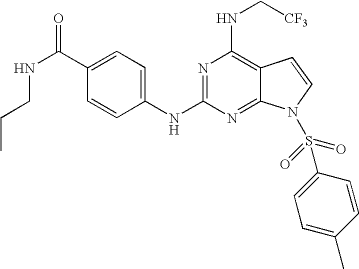

To 4-amino-N-propylbenzamide (36.7 g) was added 2-chloro-7-[(4-methylphenyl)sulfonyl]-N-(2,2,2-trifluoroethyl)-7H-pyrrolo[2,3-d]pyrimidin-4-amine (69.4 g), solid potassium carbonate (34.4 g) and nitrogen-purged tert-butanol (1700 ml). This mixture was purged with nitrogen for 10 min, tris(dibenzylideneacetone)dipalladium (3.14 g) and 2-dicyclohexylphosphino-2′,4′,6′-triisopropyl′biphenyl (3.28 g) were added. The mixture was heated at 85° C. overnight under nitrogen, excluding light. The reaction was cooled, partitioned between ethyl acetate and water, the organic phase washed with water, brine, dried and evaporated in vacuo to a dark red oil/foam. This crude product was dissolved in warm ethyl acetate (500 ml) and cyclohexane (500 ml) gradually added. The resulting solid was isolated by filtration under vacuum and the isolated beige solid washed with cyclohexane. The sticky solid was dissolved in ethyl acetate and evaporated to give a solid which was purified by chromatography on silica (1.5 kg), eluting with an ethyl acetate/DCM gradient (0-50%). Evaporation of the solvents from the appropriate fractions gave the desired product as a white foam (70.76 g). LC/MS; Rt 3.54 min, MH⁺ 547. NMR; [D₆-DMSO] δH 9.51, (1H, s), 8.34-8.29, (2H, m), 7.99-7.96, (4H, m), 7.84, (2H, d), 7.37-7.36, (3H, m), 6.85, (1H, d), 4.36, (2H, m), 3.23, (2H, q), 2.31, (3H, s), 1.55, (2H, m), 0.90, (3H, t) plus ethyl acetate.

### Intermediate 17

### 2-Chloro-7-[(4-methyl phenyl)sulfonyl]-N-(2,2,2-trifluoroethyl)-7H-pyrrolo[2,3-d]pyrimidin-4-amine

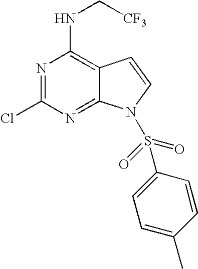

To 2,4-dichloro-7-[(4-methylphenyl)sulfonyl]-7H-pyrrolo[2,3-d]pyrimidine (140 g) suspended in ethanol (1900 ml) was added DIPEA (105.9 g) followed by trifluoroethylamine (81.2 g). The mixture was heated to reflux, using a dry-ice condenser on top of the water condenser. After 4.5 h trifluoroethylamine (33 ml) was added. The reaction was stirred at 75° C. overnight. Trifluoroethylamine (33 ml) was added after slight cooling and heating continued. After 23.5 h the reaction was cooled and the volatiles evaporated. The resulting oil was dissolved in ethyl acetate (1100 ml), washed with water, brine, dried and evaporated to a brown oil that solidified overnight. This slightly waxy solid was crushed and stirred well in ether (350 ml) for 15 min. Hexane was added (300 ml) and the slurry filtered under vacuum. The solid was washed with ether/hexane (1:1, 300 ml) and sucked dry before being dried under high vacuum to give the desired product as a pale yellow-beige solid (111.4 g). LC/MS; Rt 3.56 min, MH⁺ 405. NMR; [D₆-DMSO] δH 8.90, (1H, m), 7.96, (2H, d), 7.65, (1H, d), 7.46, (2H, d), 6.96, (1H, d), 4.30, (2H, m), 2.37, (3H, s).

The filtrate from the first crop was evaporated and re-worked as above (×2) to give a second crop of product, (27.59 g).

### Intermediate 18

### 4-Amino-N-propylbenzamide

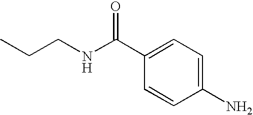

To palladium on carbon (10%, 50% wet, 4 g) was added ethyl acetate (100 ml) followed by the nitroamide (100 g, Butt Park) in ethyl acetate (1600 ml) and the mixture hydrogenated at room temperature and atmospheric pressure overnight. The reaction was filtered and the catalyst washed with ethyl acetate. The filtrate and washings were dried (magnesium sulphate), filtered and evaporated, to give the desired product as a pale gold oil which was further dried under vacuum for 1 h (89.0 g). LC/MS; Rt 1.85 min, MH⁺ 179. NMR; [D₆-DMSO] δH 7.96, (1H, t), 7.56, (2H, d), 6.52, (2H, d), 5.56, (2H, brs), 3.15, (2H, q), 1.49, (2H, m), 0.86, (3H, t).

### Example 25

### N-Propyl-4-({4-[(2,2,2-trifluoroethyl)amino]-1H-pyrrolo[2,3-d]pyrimidin-2-yl}amino)benzamide 4-methylbenzenesulfonate

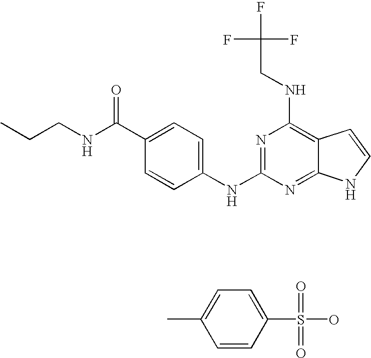

N-Propyl-4-({4-[(2,2,2-trifluoroethyl)amino]-1H-pyrrolo[2,3-d]pyrimidin-2-yl}amino)benzamide (61.5 g) was suspended in dry THF (1050 ml) and the mixture stirred at 40° C. under nitrogen. A solution of p-toluene sulphonic acid monohydrate (29.8 g, Aldrich) in dry THF (185 ml) was added dropwise. After the first 50 ml had been added the mixture was seeded with a little N-propyl-4-({4-[(2,2,2-trifluoroethyl)amino]-1H-pyrrolo[2,3-d]pyrimidin-2-yl}amino)benzamide 4-methylbenzenesulfonate. The remainder of the p-toluene sulphonic acid was added dropwise over ˜45 min keeping the reaction temperature at ˜40° C. After addition was complete the reaction mixture was stirred at 40° C. for a further 1 h, cooled to 0° C. over 2 h, held at 0° C. for 0.5 h, then warmed to ambient over 0.5 h. The crystals were filtered off, washed with THF (500 ml), and dried in vacuo at 40° C. overnight. The crystals were ground and re-dried at 40° C. for a further night to yield the desired product (87.5 g). NMR; [D₆-DMSO] δH 11.71, (1H, s), 9.78, (1H, brs), 8.94, (1H, brs), 8.35, (1H, t), 7.83, (2H, d), 7.74, (2H, d), 7.51, (2H, d), 7.13, (2H, d), 7.02, (1H, s), 6.68, (1H, s), 4.41, (2H, m), 3.31, (2H, q), 2.29, (3H, s), 1.53, (2H, m), 0.89, (3H, t).

### Example 26

### 4-({4-[(1-Methylethyl)amino]-7H-pyrrolo[2,3-d]pyrimidin-2-yl}amino)benzamide

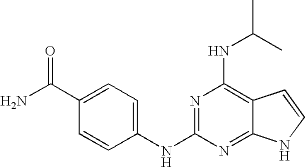

To 4-({4-[(1-methylethyl)amino]-7-[(4-methylphenyl)sulfonyl]-7H-pyrrolo[2,3-d]pyrimidin-2-yl}amino)benzamide (50.52 g) in methanol (1250 ml) was added anhydrous potassium carbonate (45 g) and water (250 ml). The suspension was heated to reflux. After 4.75 h the reaction was cooled, the methanol evaporated in vacuo and the aqueous residue extracted with ethyl acetate (1 l, then 3×100 ml). The combined organics were washed with brine, dried (magnesium sulphate), filtered and the solvents evaporated to give a brown foam. This was purified by chromatography on silica (1 kg), eluting with ethyl acetate and then with increasing percentages of methanol (0-5%), to give, after evaporation of the solvents from the appropriate fractions, the desired product as a slightly green foam (32.3 g). LC/MS; Rt 2.21 min, MH⁺ 311.

This material was dissolved with warming in acetone (400 ml), water was added slowly until the mixture remained cloudy (total vol ˜1.31). Scratching initiated crystals, the mixture was left un-stoppered for 3 days, cooled in an ice-bath for ˜2 h and the crystals isolated by filtration. The solid was washed with a little water and then dried under high vacuum at 40° C. overnight to give a pale yellowish solid (27.8 g). LC/MS; Rt 2.25 min, MH⁺ 311. NMR; [D₆-DMSO] δH 11.03, (1H, s), 8.93, (1H, s), 7.91, (2H, d), 7.74, (2H, d), 7.72, (1H, br s), 7.04, (2H, br s), 6.80, (1H, s), 6.47, (1H, s), 4.44, (1H, m), 1.25, (6H, d) and acetone.

### Intermediate 19

### 4-({4-[(1-Methylethyl)amino]-7-[(4-methyl phenyl)sulfonyl]-7H-pyrrolo[2,3-d]pyrimidin-2-yl}amino)benzamide

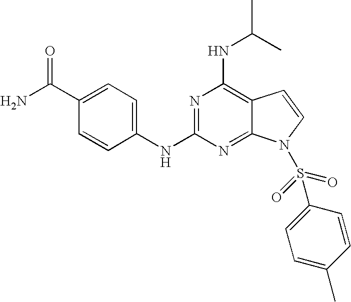

To 2-chloro-N-(1-methylethyl)-7-[(4-methylphenyl)sulfonyl]-7H-pyrrolo[2,3-d]pyrimidin-4-amine (45 g) was added 4-aminobenzamide (20.1 g), nitrogen-purged tert-butanol (1125 ml), anhydrous potassium carbonate (24.7 g), 2-dicyclohexylphosphino-2′,4′,6′-triisopropylbiphenyl (2.35 g) and tris(dibenzylidene acetone)dipalladium (2.26 g). The mixture was heated to reflux under nitrogen, excluding light. After 5.5 h the mixture was cooled slightly and the solvent evaporated to leave red-brown oil/foam. This residue was diluted with water (1000 ml) and extracted with ethyl acetate. The combined organics were washed with brine, dried (magnesium sulphate), filtered through Celite and the solvent evaporated to leave a red-brown oil/foam. This material was purified by column chromatography on silica (1600 g), eluting with DCM/ethyl acetate (2:1 through to 1:1 and finally with 2:3). Evaporation of the solvents from the appropriate fractions gave the desired compound as a pale pink-beige solid (42.1 g). LC/MS; Rt 3.32 min, MH⁺ 465. NMR; [D₆-DMSO] δH 9.31, (1H, s), 7.97, (4H, d), 7.83, (2H, d), 7.80, (1H, br s), 7.47, (1H, d), 7.38, (2H, d), 7.28, (1H, d), 7.11, (1H, br s), 6.80, (1H, d), 4.37, (1H, m), 2.31, (3H, s), 1.21, (6H, d) and ethyl acetate.

### Example 27

### 4-({4-[(1,1-Dimethylethyl)amino]-1H-pyrrolo[2,3-d]pyrimidin-2-yl}amino)-N-methylbenzamide trifluoroacetate

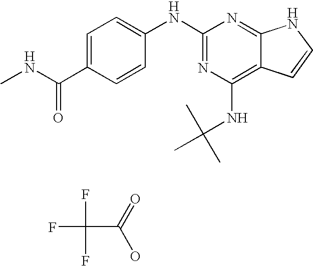

N-(1,1-dimethylethyl)-2-iodo-7-[(4-methylphenyl)sulfonyl]-7H-pyrrolo[2,3-d]pyrimidin-4-amine (0.8 mmol) was dissolved in DMF (16 ml). Bis(dibenzylideneacetone) palladium (10 mol %, Aldrich), 2-dicyclohexylphosphino-2′-(N,N-dimethylamino) biphenyl (15 mol %), cesium carbonate (0.3 mmol) and 4-amino-N-methylbenzamide (0.15 mmol) were combined with an aliquot of this solution (2 ml). The reaction was heated at 80° C. for 2 h, allowed to cool, filtered through Celite and concentrated. The reaction was dissolved in methanol (1.5 ml), treated with sodium methoxide in methanol (0.5M, 500 μl), stirred at 70° C. for 2 h and left to stand at room temperature overnight. The reaction was heated for a further 5 h, concentrated and purified using MDAP. The fractions containing product were evaporated to dryness to give title compound (3 mg). LC/MS; Rt 2.58 min, MH⁺ 339.

### Intermediate 20

### N-(1,1-Dimethylethyl)-2-iodo-7-[(4-methylphenyl)sulfonyl]-7H-pyrrolo[2,3-d]pyrimidin-4-amine

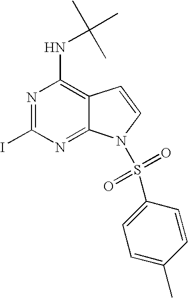

4-Chloro-2-iodo-7-[(4-methylphenyl)sulfonyl]-7H-pyrrolo[2,3-d]pyrimidine (500 mg) in ethanol (10 ml) was treated with tert-butylamine (610 μl) and DIPEA (410 μl). The reaction was stirred at 80° C. for 6.5 h and left to stand at room temperature over the weekend. tert-Butylamine (100 μl) was added and the reaction heated at 80° C. for 2 h. The reaction was concentrated and purified by chromatography on a silica cartridge (50 g) eluting with an ethyl acetate/cyclohexane gradient (0-100%) over 30 mins. The appropriate fractions were combined and reduced to dryness to leave title compound (0.4 g). LC/MS; Rt 4.01 min, MH⁺ 471.

### Example 28

### N-(1-Methylethyl)-4-({4-[(2,2,2-trifluoroethyl)amino]-1H-pyrrolo[2,3-d]pyrimidin-2-yl}amino)benzamide

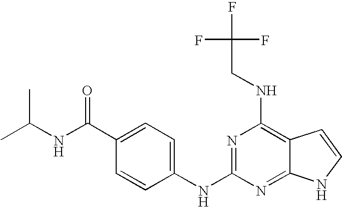

The 4-({7-[(4-methylphenyl)sulfonyl]-4-[(2,2,2-trifluoroethyl)amino]-7H-pyrrolo[2,3-d]pyrimidin-2-yl}amino)benzoic acid (60 mg), TBTU (42 mg) and DIPEA (0.062 ml) in DMF (0.75 ml) were stirred at room temperature in a stoppered flask. After 30 min isopropylamine (0.101 ml) was added and the reaction stirred for 1 h. The reaction was reduced to dryness in vacuo and the residue azeotroped with methanol. The residue, dissolved in methanol, was applied to a pre-conditioned SCX-2 cartridge (5 g), which was washed with methanol and the product eluted with 2N ammonia in methanol. The basic fraction was reduced to dryness, the residue dissolved in water (0.5 ml) and methanol (1.5 ml) and potassium carbonate (41 mg) added. The mixture was stirred at 85° C. for 6 h. Potassium carbonate (30 mg) was added and the reaction stirred at 85° C. for a further 15 h. The reaction was filtered, the solid washed with water and ether and dried in vacuo, to give the title compound as an off-white solid (17 mg). LC/MS; MH⁺ 393, Rt 3.03 min.

### Intermediate 21

### 4-({7-[(4-Methyl phenyl)sulfonyl]-4-[(2,2,2-trifluoroethyl)amino]-7H-pyrrolo[2,3-d]pyrimidin-2-yl}amino)benzoic acid

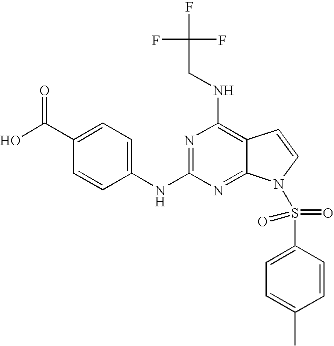

The 1,1-dimethylethyl 4-({7-[(4-methylphenyl)sulfonyl]-4-[(2,2,2-trifluoroethyl)amino]-7H-pyrrolo[2,3-d]pyrimidin-2-yl}amino)benzoate (150 mg), in DCM (6 ml) was treated with TFA (1 ml) and stirred at room temperature for 1.75 h. The volatiles were evaporated under vacuum and the residual solid dissolved in ethyl acetate (25 ml). The solution was washed with water (2×25 ml) and dried (hydrophobic frit). Evaporation of the solvent left the title compound as a green solid (130 mg). LC/MS; MH⁺ 506, Rt 3.72 min.

### Intermediate 22

### 1,1-Dimethylethyl-4-({7-[(4-methyl phenyl)sulfonyl]-4-[(2,2,2-trifluoroethyl)amino]-7H-pyrrolo[2,3-d]pyrimidin-2-yl}amino)benzoate

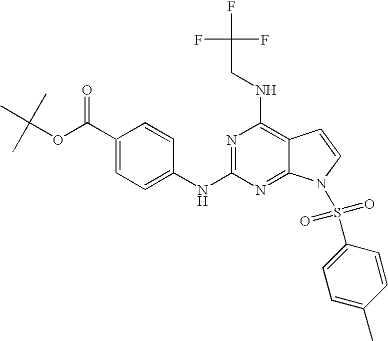

A mixture of 2-chloro-7-[(4-methylphenyl)sulfonyl]-N-(2,2,2-trifluoroethyl)-7H-pyrrolo[2,3-d]pyrimidin-4-amine (200 mg), 2-dicyclohexylphosphino-2′,4′,6′-triisopropyl biphenyl (11.8 mg), tris(dibenzylideneacetone)dipalladium (0) (45.2 mg), potassium carbonate (95.6 mg) and tert-butyl 4-aminobenzoate (114.5 mg, Fluka) in t-butanol (5 ml) was degassed. The vessel was sealed and irradiated at 120° C. for 3 h in a microwave. The reaction mixture was reduced to dryness and the residue suspended in ethyl acetate. The suspension was applied to a SCX-2 cartridge (10 g, pre-conditioned with methanol followed by ethyl acetate) and eluted with ethyl acetate, methanol and 2N ammonia in methanol. The ammonia fraction was concentrated, re-dissolved in methanol and adsorbed onto Florisil. This was purified by chromatography on a silica cartridge (100 g), eluting with an ethyl acetate/cyclohexane gradient (0-50%). The appropriate fractions were combined, reduced to dryness and azeotroped with ether to give the title compound as a yellow solid (150 mg). LC/MS; MH⁺ 562, Rt 4.00 min.

### Example 29

### N-(2-Methylpropyl)-4-({4-[(2,2,2-trifluoroethyl)amino]-1H-pyrrolo[2,3-d]pyrimidin-2-yl}amino)benzamide

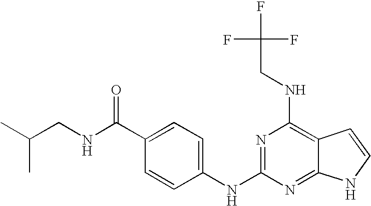

The 4-({7-[(4-methylphenyl)sulfonyl]-4-[(2,2,2-trifluoroethyl)amino]-7H-pyrrolo[2,3-d]pyrimidin-2-yl}amino)benzoic acid (60 mg), TBTU (42 mg) and DIPEA (0.062 ml) in DMF (0.75 ml) were stirred at room temperature in a stoppered flask. After 30 min isobutylamine (0.117 ml) was added and the mixture stirred for 1 h. The solvent was evaporated under vacuum and the residue azeotroped with methanol. The residue in methanol was applied to a pre-conditioned SCX-2 cartridge (5 g), the cartridge washed with methanol and the product eluted with 2N ammonia in methanol. The basic fraction was reduced to dryness and the residue dissolved in water (0.5 ml) and methanol (1.5 ml). Potassium carbonate (69 mg) was added and the mixture stirred at 85° C. for 7 h. The mixture was filtered and the solid washed with water and ether. The washes were repeated and the ether fractions were combined with the solid and reduced to dryness. The residual solid was dissolved in warm methanol and applied to a SCX-2 cartridge (5 g, pre-conditioned with methanol). The cartridge was washed with methanol and the product eluted with 2N ammonia in methanol solution. The ammonia fraction was reduced to dryness to leave the title compound as a white solid (23.2 mg). LC/MS; MH⁺ 407, Rt 3.09 min.

### Example 30

### N-Ethyl-4-({4-[(2,2,2-trifluoroethyl)amino]-1H-pyrrolo[2,3-d]pyrimidin-2-yl}amino)benzamide

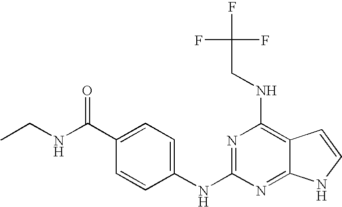

N-Ethyl-4-({7-[(4-methylphenyl)sulfonyl]-4-[(2,2,2-trifluoroethyl)amino]-7H-pyrrolo[2,3-d]pyrimidin-2-yl}amino)benzamide (306 mg) and potassium carbonate (794 mg) in methanol (10 ml) and water (5 ml) were stirred at 85° C. for 2.5 h. The reaction was allowed to cool to ambient temperature and the solvents evaporated under vacuum. The solid was suspended in methanol, filtered and the filtrate applied to an SCX-2 cartridge (20 g, pre-conditioned with methanol). The cartridge was washed with methanol the product eluted with 2N ammonia in methanol. The ammonia fractions were combined and reduced to dryness. The residual solid was dissolved in methanol and absorbed onto Florisil. This material was purified by chromatography on a silica cartridge (50 g), eluting with a DCM/methanol gradient (0-25%) over 30 min. A precipitate formed in one of the eluted fractions, this was isolated by filtration and washed with DCM. After drying in vacuo, this yielded the title compound as a white/pink solid (12 mg). LC/MS; MH⁺ 379, Rt 2.81 min.

### Intermediate 23

### N-Ethyl-4-({7-[(4-methylphenyl)sulfonyl]-4-[(2,2,2-trifluoroethyl)amino]-7H-pyrrolo[2,3-d]pyrimidin-2-yl}amino)benzamide

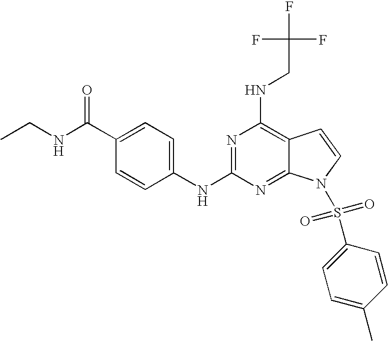

The 2-chloro-7-[(4-methylphenyl)sulfonyl]-N-(2,2,2-trifluoroethyl)-7H-pyrrolo[2,3-d]pyrimidin-4-amine (350 mg), 2-dicyclohexylphosphino-2′,4′,6′-triisopropyl biphenyl (21 mg), tris(dibenzylideneacetone)dipalladium (0) (48 mg), potassium carbonate (167 mg) and 4-(ethylcarbamyl)aniline (170 mg, Butt Park) in t-butanol (12 ml) were heated at 80° C. under nitrogen for overnight. The reaction was removed from the heat source and the contents transferred to a microwave vessel. The mixture was degassed, tris(dibenzylideneacetone)dipalladium (0) (48 mg) was added. The mixture was irradiated in a sealed vessel by microwave at 105° C. for 2 h. The reaction mixture was degassed under nitrogen and heated in the microwave again at 105° C. for 1.5 h. The reaction mixture was concentrated in vacuo and the residual solid suspended in ethyl acetate. After filtration through Celite, the filtrate was pre-absorbed onto Florisil and purified by chromatography on a silica cartridge (100 g), eluting with an ethyl acetate/cyclohexane gradient (0-100%) over 60 min. Appropriate fractions were combined and evaporated to give the title compound as a yellow oil (306 mg). LC/MS; MH⁺ 533, Rt 3.61 min.

### Example 31

### N,N-Dimethyl-4-({4-[(2,2,2-trifluoroethyl)amino]-1H-pyrrolo[2,3-d]pyrimidin-2-yl}amino)benzamide

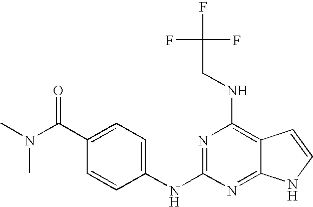

The N,N-dimethyl-4-({7-[(4-methylphenyl)sulfonyl]-4-[(2,2,2-trifluoroethyl)amino]-7H-pyrrolo[2,3-d]pyrimidin-2-yl}amino)benzamide (114 mg) in IPA (3 ml) was treated with aqueous sodium hydroxide (2N, 0.64 ml) and heated at 80° C. for 6 h. The temperature was lowered to 70° C. and the reaction stirred overnight. The reaction mixture was cooled to room temperature after 22 h of heating and the solvents evaporated under vacuum. The residue was suspended in ethyl acetate and applied to an SCX-2 cartridge (5 g, pre-conditioned with methanol and ethyl acetate). The cartridge was washed with ethyl acetate, methanol and the product eluted with 2N ammonia in methanol. The solvent was evaporated from the ammonia fraction, and the residual oil was dissolved in methanol and adsorbed onto Florisil. This material was purified by chromatography on a silica cartridge (20 g), eluting with a gradient of ethyl acetate/methanol (1:1) in cyclohexane (10-100%). The appropriate fractions were combined and the solvents evaporated to leave a brown solid. Trituration with ether and drying under nitrogen gave the title compound as a yellow/brown solid (37.2 mg). LC/MS; MH⁺ 379, Rt 2.64 min.

### Intermediate 24

### N,N-Dimethyl-4-({7-[(4-methylphenyl)sulfonyl]-4-[(2,2,2-trifluoroethyl)amino]-7H-pyrrolo[2,3-d]pyrimidin-2-yl}amino)benzamide

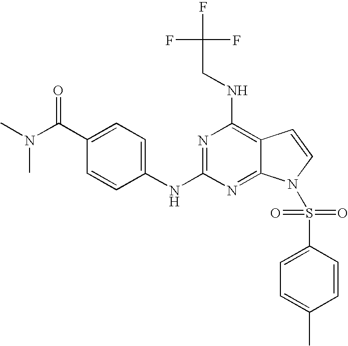

2-Chloro-7-[(4-methylphenyl)sulfonyl]-N-(2,2,2-trifluoroethyl)-7H-pyrrolo[2,3-d]pyrimidin-4-amine (117 mg), 4-(N,N-dimethylcarbamoyl)aniline (57 mg, Apollo Scientific Ltd), tris(dibenzylideneacetone)dipalladium (0) (16 mg), 2-dicyclohexylphosphino-2′,4′,6′-triisopropyl biphenyl (6.9 mg) and potassium carbonate (55.9 mg) in t-butanol (2 ml) was heated at 120° C. in a sealed vessel by microwave irradiation for 1 h. The reaction mixture was diluted with ethyl acetate and filtered through a pad of Celite. The filtrate was applied to an SCX-2 cartridge (5 g, pre-conditioned with methanol and ethyl acetate. The cartridge was washed with ethyl acetate, methanol and the product eluted with 2N ammonia in methanol solution. The ammonia fraction was reduced to dryness under vacuum and adsorbed onto Florisil from methanol. This was purified by chromatography on a silica cartridge (20 g), eluting with an ethyl acetate/cyclohexane gradient (25-100%). Appropriate fractions were combined, the solvents evaporated and azeotroped with ether to obtain the title compound as a glassy solid (114 mg). LC/MS; MH⁺ 533, Rt 3.41 min.

### Example 32

### N-(Cyclopropyl methyl)-4-({4-[(2,2,2-trifluoroethyl)amino]-1H-pyrrolo[2,3-d]pyrimidin-2-yl}amino)benzamide

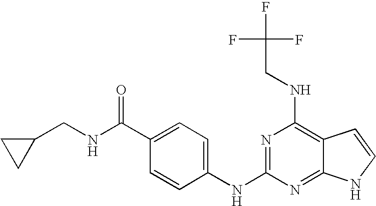

2-Chloro-7-[(4-methylphenyl)sulfonyl]-N-(2,2,2-trifluoroethyl)-7H-pyrrolo[2,3-d]pyrimidin-4-amine (100 mg), 4-amino-N-(cyclopropylmethyl)benzamide hydrochloride (62.8 mg) tris(dibenzylideneacetone)dipalladium (0) (13.6 mg), 2-dicyclohexylphosphino-2′,4′,6′-triisopropyl biphenyl (5.9 mg) and potassium carbonate (91.8 mg) in t-butanol (1.5 ml) was stirred and irradiated at 120° C. in a sealed vessel in a microwave for 1 h. The mixture was heated for a further 1 h at 150° C. Tris(dibenzylideneacetone)dipalladium (0) (7 mg) and potassium carbonate (17 mg) were added to the reaction. The vessel was sealed and the mixture heated at 150° C. for 45 min in the microwave. The reaction mixture was diluted with ethyl acetate (2 ml) and filtered through Celite. The filtrate was applied to an SCX-2 cartridge (5 g, pre-conditioned with methanol and ethyl acetate). The cartridge was washed with ethyl acetate, methanol and the product eluted with 2N ammonia in methanol solution. The ammonia fraction was reduced to dryness under reduced pressure and the residue dissolved in IPA (1.5 ml). The solution was treated with aqueous sodium hydroxide (2N, 1 ml) and the mixture stirred at 80° C. for 16 h. The solvents were evaporated under a stream of nitrogen and the residue suspended in methanol. The suspension was applied to an SCX-2 cartridge (2 g, pre-conditioned with methanol). The solid retained on top of the cartridge was dried under nitrogen to obtain the title compound as an off-white solid (33 mg). LC/MS; MH⁺ 405, Rt 2.89 min.

### Intermediate 25

### 4-Amino-N-(cyclopropylmethyl)benzamide hydrochloride

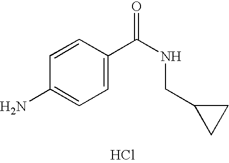

N-(Cyclopropylmethyl)-4-nitrobenzamide (23.8 g) was dissolved in ethanol and hydrogenated over palladium on carbon (10%, 1.8 g). The reaction was filtered, the ethanol evaporated in vacuo and the residual gum partitioned between ethyl acetate and sodium bicarbonate solution. The organic phase was reduced to dryness in vacuo and hydrochloric acid in dioxane (4N) added. The white solid was isolated by filtration, washed with ether and dried in vacuo to obtain the title compound (15.5 g). NMR; [D₆-DMSO] δH 9-8, (3H, bm), 7.81, (2H, d), 7.11, (2H, d), 3.12, (2H, m), 1.01, (1H, m), 0.42, (2H, m), 0.22 (2H, m).

### Intermediate 26

### N-(Cyclopropyl methyl)-4-nitrobenzamide

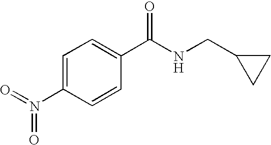

4-Nitrobenzoyl chloride (20 g, Aldrich) was dissolved in DCM (500 ml) and triethylamine (16.5 ml) added. Cyclopropanemethylamine (21 ml, Aldrich) was added (exothermic) and the reaction stirred at room temperature under nitrogen overnight. The volatiles were evaporated and the residue dried in vacuo to give the title compound. LC/MS; MH⁺ 221, Rt 2.7 min.

### Example 33

### N²-{4-[(4-Methyl-1-piperazinyl)carbonyl]phenyl}-N⁴-(2,2,2-trifluoroethyl)-1H-pyrrolo[2,3-d]pyrimidine-2,4-diamine

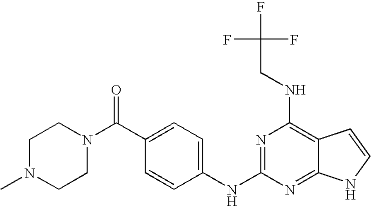

2-Chloro-7-[(4-methylphenyl)sulfonyl]-N-(2,2,2-trifluoroethyl)-7H-pyrrolo[2,3-d]pyrimidin-4-amine (100 mg), 1-(4-Aminobenzoyl)-4-methylpiperazine (65.1 mg, Butt Park Ltd), tris(dibenzylideneacetone)dipalladium (0) (13.6 mg), 2-dicyclohexylphosphino-2′,4′,6′-triisopropyl biphenyl (5.9 mg) and potassium carbonate (47.8 mg) in t-butanol (1.5 ml) was stirred and irradiated at 120° C. in a sealed vessel by microwave for 1 h. The mixture was heated for a further 30 min at 115° C. The reaction mixture was diluted with ethyl acetate (2 ml) and filtered through Celite. The filtrate was applied to an SCX-2 cartridge (5 g, pre-conditioned with methanol and ethyl acetate). The cartridge was washed with ethyl acetate, methanol and the product eluted with 2N ammonia in methanol solution. The ammonia fraction was reduced to dryness in vacuo and the residue dissolved in IPA (1.5 ml). The solution was treated with aqueous sodium hydroxide (2N, 1 ml) and stirred at 80° C. for 16 h. The solvents were evaporated under a stream of nitrogen and the residue suspended in methanol. The suspension was applied to an SCX-2 cartridge (2 g, pre-conditioned with methanol). The product was eluted in the methanol wash which was concentrated under vacuum. The residue was purified on MDAP and the appropriate fractions combined and evaporated. The sample was adsorbed from methanol onto Florisil and applied to a silica cartridge (20 g). This was eluted with a gradient of ethyl acetate/methanol (1:1) in cyclohexane (10-100%). Appropriate fractions were combined, the solvents evaporated to obtain the title compound. LC/MS; MH⁺ 434, Rt 2.03 min.

Method 6:

The 4-({4-[(2,2,2-trifluoroethyl)amino]-1H-pyrrolo[2,3-d]pyrimidin-2-yl}amino)benzoic acid (527 mg) in anhydrous DMF (3.75 ml) was treated with DIPEA (0.78 ml) and TBTU (530 mg) and left at room temperature for 30 min. A portion (0.302 ml) of this solution was added to a solution of amine (0.20 mM) in DMF (0.25 ml). The reaction mixture was left at room temperature under nitrogen overnight. The mixture was reduced to dryness in a vacuum centrifuge and the residue purified by MDAP. The appropriate fractions were evaporated using a vacuum centrifuge to give the desired products.

The following compounds were prepared using Method 6:

| | | | | | LC/MS |
|---|---|---|---|---|---|
| | | | Amine/ | LC/MS | Rt |
| Example | Structure | Name | Source | MH⁺ | (min) |
| 34 | 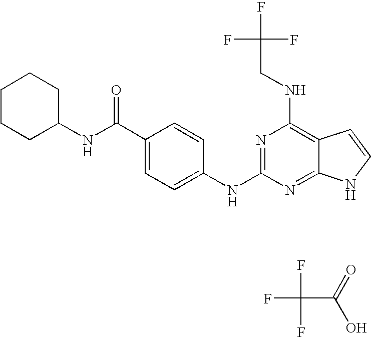 | N-cyclohexyl-4-({4-[(2,2,2- trifluoroethyl)amino]- 1H-pyrrolo[2,3-d]pyrimidin- 2-yl}amino)benzamide trifluoroacetate | Cyclohexylamine/ Aldrich | 433 | 3.2 |
| 35 | 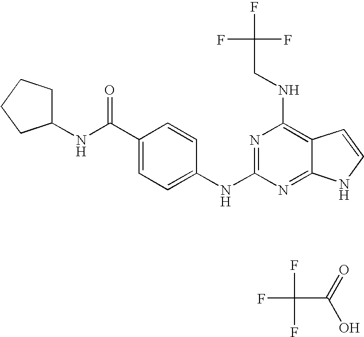 | N-cyclopentyl-4-({4-[(2,2,2- trifluoroethyl)amino]- 1H-pyrrolo[2,3-d]pyrimidin- 2-yl}amino)benzamide trifluoroacetate | Cyclopentylamine/ Aldrich | 419 | 3.07 |
| 36 | 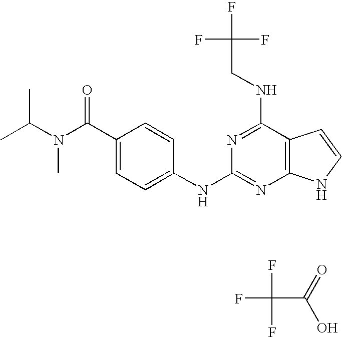 | N-methyl-N-(1-methylethyl)- 4-({4-[(2,2,2-trifluoroethyl) amino]-1H-pyrrolo[2, 3-d]pyrimidin-2-yl}amino) benzamide trifluoroacetate | Methylisopropylamine/ Aldrich | 407 | 2.84 |
| 37 | 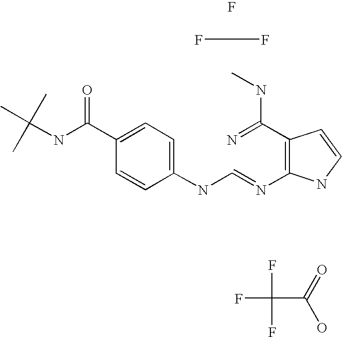 | N-(1,1-dimethyl-4- {4-(2,2,2-trifluoroethyl) amino]-1H-pyrrolo[2, 3-d]pyrimidin-2-yl}amino) benzamide trifluoroacetate | t-Butylamine/ Aldrich | 407 | 3.06 |
| 38 | 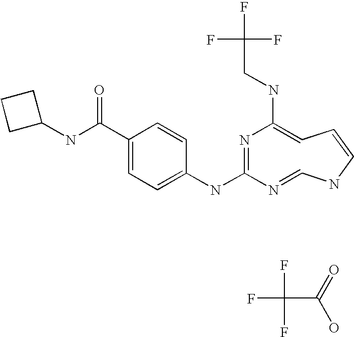 | N-cyclobutyl-4-({4-[(2,2,2- trifluoroethyl)amino]-1H- pyrrolo[2,3-d]pyrimidin-2- yl}amino)benzamide trifluoroacetate | Cyclobutylamine/ Aldrich | 405 | 2.96 |

### Intermediate 27

### 4-({4-[(2,2,2-Trifluoroethyl)amino]-1H-pyrrolo[2,3-d]pyrimidin-2-yl}amino)benzoic acid

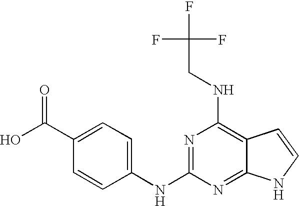

Ethyl 4-({7-[(4-methylphenyl)sulfonyl]-4-[(2,2,2-trifluoroethyl)amino]-7H-pyrrolo[2,3-d]pyrimidin-2-yl}amino)benzoate (2.5 g) was suspended in ethanol (60 ml) and treated with aqueous sodium hydroxide (2N, 14.1 ml). The mixture was stirred at 80° C. for 3 h and allowed to cool to ambient temperature. The reaction mixture was acidified with glacial acetic acid while stirring in an ice bath. The precipitate was isolated by filtration, dissolved in acetone, filtered and the acetone evaporated. The residue was recrystallised from ethyl acetate plus drops of acetone and water. The precipitated solid was isolated by filtration to give the title compound (1.0 g). LC/MS; MH⁺ 351.98, Rt 2.87 min.

### Intermediate 28

### Ethyl 4-({7-[(4-methylphenyl)sulfonyl]-4-[(2,2,2-trifluoroethyl)amino]-7H-pyrrolo[2,3-d]pyrimidin-2-yl}amino)benzoate

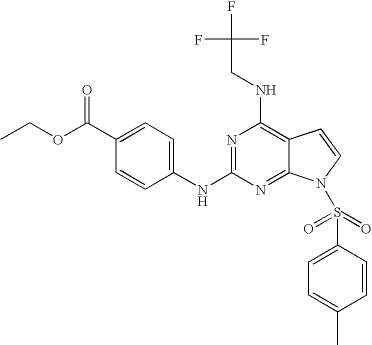

2-Chloro-7-[(4-methylphenyl)sulfonyl]-N-(2,2,2-trifluoroethyl)-7H-pyrrolo[2,3-d]pyrimidin-4-amine (7.5 g), ethyl-4-aminobenzoate (3.37 g, Aldrich), tris(dibenzylideneacetone)dipalladium (0) (510 mg), 2-dicyclohexylphosphino-2′,4′,6′-triisopropyl biphenyl (142 mg) and potassium carbonate (3.59 g) in t-butanol (40 ml) was heated at 110° C. under nitrogen overnight. The solvent was evaporated in vacuo and the residue dissolved in ethyl acetate. The solution was filtered through Celite and the filtrate reduced to dryness. The residue was crystallised from ethanol and the crystals isolated by filtration. These were dissolved in boiling ethanol, the solution allowed to cool initially to room temperature and then at ˜4° C. for 5 h. The ethanol was drained from the resulting crystals, which were then further dried on a sinter to give the title compound (4.7 g). LC/MS: MH⁺ 533.98, Rt 3.84 min.

Method 7:

The 4-({4-[(2,2,2-trifluoroethyl)amino]-1H-pyrrolo[2,3-d]pyrimidin-2-yl}amino)benzoic acid (842 mg) in anhydrous DMF (6 ml) was treated with TBTU (847.2 mg) DIPEA (1.7 ml) and left at room temperature for 30 min. A portion of this solution (0.32 ml) was added to a solution of amine (0.20 mM) in DMF (0.25 ml). The reaction mixture was left at room temperature for 3.5 days. Further TBTU (32 mg) and DIPEA (0.017 ml) was added and the reaction left at room temperature overnight. The reaction was purified by MDAP and appropriate fractions combined and evaporated to give the desired product.

The following compounds were prepared using Method 7:

| | | | | | LC/MS |
|---|---|---|---|---|---|
| | | | Amine/ | LC/MS | Rt |
| Example | Structure | Name | Source | MH⁺ | (min) |
| 39 | 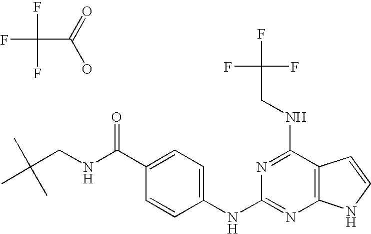 | N-(2,2-dimethylpropyl)-4- ({4-[(2,2,2-trifluoroethyl) amino]-1H-pyrrolo[2,3-d]pyrimidin- 2-yl}amino)benzamide trifluoroacetate | Neopentylamine/ Fluorochem Ltd | 420.89 | 3.16 |
| 40 | 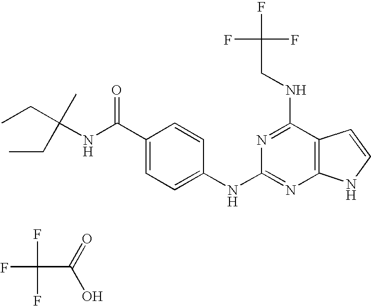 | N-(1-ethyl-1-methylpropyl- 4-({4-[(2,2,2-trifluoroethyl) amino]-1H-pyrrolo[2,3-d]pyrimidin-2-yl}amino) benzamide trifluoroacetate | 3-Methyl-3- pentanamine/ Matrix Scientific | 435 | 3.30 |
| 41 | 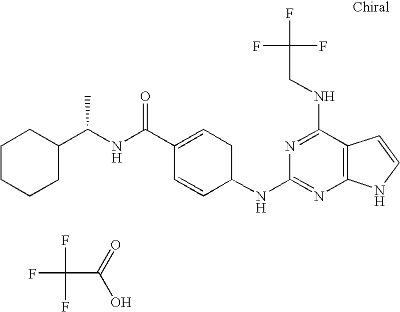 | N-[(1S)-1-cyclohexylethyl]- 4-({4-[(2,2,2-trifluoroethyl) amino]-1H-pyrrolo[2,3-d]pyrimidin-2-yl}amino) benzamide trifluoroacetate | S(+)-1- Cyclohexylethylamine/ Aldrich | 461 | 3.41 |
| 42 | 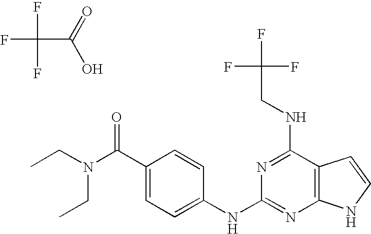 | N,N-diethyl-4-({4-[(2,2,2- trifluoroethyl)amino]-1H- pyrrolo[2,3-d]pyrimidin-2-yl}amino)benzamide trifluoroacetate | Diethylamine/ Aldrich | 406.9 | 2.89 |

### Example 43

### 4-({4-[(2,2,2-Trifluoroethyl)amino]-1H-pyrrolo[2,3-d]pyrimidin-2-yl}amino)benzamide

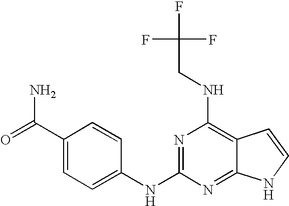

4-({7-[(4-Methylphenyl)sulfonyl]-4-[(2,2,2-trifluoroethyl)amino]-7H-pyrrolo[2,3-d]pyrimidin-2-yl}amino)benzamide (229 mg) was dissolved in sodium methoxide in methanol (0.5M, 3 ml) and the solution heated at 80° C. under nitrogen for ˜1 h and then left overnight at room temperature. The reaction was diluted with water (˜5 ml) and the precipitate isolated by filtration. The solid was washed with water, sucked dry on the sinter and further dried at 45° C. under vacuum to give the desired product as a cream solid (133 mg). LC/MS; MH⁺ 350.93, Rt 2.51 min.

### Intermediate 29

### 4-({7-[(4-Methyl phenyl)sulfonyl]-4-[(2,2,2-trifluoroethyl)amino]-7H-pyrrolo[2,3-d]pyrimidin-2-yl}amino)benzamide

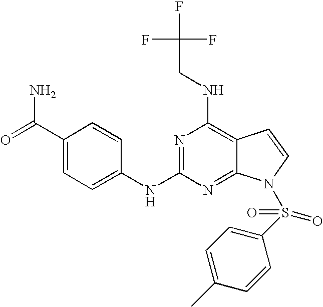

2-Chloro-7-[(4-methylphenyl)sulfonyl]-N-(2,2,2-trifluoroethyl)-7H-pyrrolo[2,3-d]pyrimidin-4-amine (200 mg), 4-aminobenzamide (81 mg), tris(dibenzylideneacetone)dipalladium (0) (12 mg), 2-dicyclohexylphosphino-2′,4′,6′-triisopropyl biphenyl (6 mg) and potassium carbonate (100 mg) were mixed in t-butanol (7.5 ml), the mixture degassed and heated at 85° C. under nitrogen for ˜20 h. Tris(dibenzylideneacetone)dipalladium (12 mg), 2-dicyclohexylphosphino-2′,4′,6′-triisopropyl biphenyl (6 mg) were added to the reaction and heating continued for 3 h at 85° C. and then at 95° C. for ˜20 h. The cooled reaction was diluted with ethyl acetate, absorbed onto silica and applied to a silica cartridge (20 g). The cartridge was eluted with an ethyl acetate/cyclohexane gradient (0-100%), the appropriate fractions combined and the solvents evaporated in vacuo to give the desired product as a pale yellow solid (230 mg). NMR; [D₆-DMSO] δH 9.51, (1H, s), 8.32, (1H, t), 7.98-7.93, (4H, m), 7.86-7.84, (3H, m), 7.40-7.37, (3H, m), 7.15, (1H, bs), 6.85, (1H, d), 4.35, (2H, m), 2.32, (3H, s).

### Example 44

### 4-{[4-(Methylamino)-1H-pyrrolo[2,3-d]pyrimidin-2-yl]amino}-N-propylbenzamide

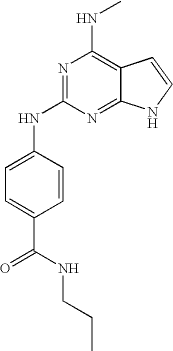

2-Chloro-N-methyl-1H-pyrrolo[2,3-d]pyrimidin-4-amine (100 mg), 4-amino-N-propylbenzamide (98 mg), tris(dibenzylideneacetone)dipalladium (0) (50.4 mg), 2-dicyclohexylphosphino-2′,4′,6′-triisopropyl biphenyl (39.1 mg), potassium carbonate (152 mg) and t-butanol (10 ml) were combined and heated by microwave in a sealed vessel at 140° C. for 40 min. The reaction was diluted with ethanol, filtered through Celite and the filtrate reduced to dryness under a stream of nitrogen. The residue was dissolved in DCM/methanol and loaded on to an SCX-2 cartridge (10 g, pre-conditioned with methanol). The cartridge was eluted with methanol and 2M ammonia in methanol. The solvent was evaporated from the methanolic ammonia fraction under a stream of nitrogen. The residue was purified by chromatography on a silica cartridge (20 g), eluting with a gradient of methanol/DCM (0-15%)+1% triethylamine over 30 min to give the title compound (41 mg). LC/MS; Rt 2.32 min, MH⁺ 325.

### Intermediate 30

### 2-Chloro-N-methyl-1H-pyrrolo[2,3-d]pyrimidin-4-amine

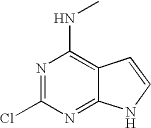

2,4-Dichloro-1H-pyrrolo[2,3-d]pyrimidine (400 mg, Pharma Lab Product List) and a solution of methylamine in ethanol (33%, 10 ml) were heated by microwave in a sealed vessel at 80° C. for 10 min. The volatiles were evaporated in vacuo and the residual solid was suspended in water and stirred for 5 min. The solid was isolated by filtration and dried under vacuum at 40° C. overnight to give the title compound (311 mg). LC/MS; Rt 2.22 min, MH⁺ 183, 185.

### Example 45

### N²-{4-[(1,1-Dioxido-4-thiomorpholinyl)carbonyl]phenyl}-N⁴-(2,2,2-trifluoroethyl)-1H-pyrrolo[2,3-d]pyrimidine-2,4-diamine

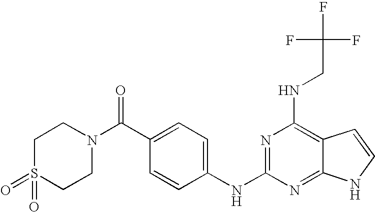

4-({4-[(2,2,2-Trifluoroethyl)amino]-1H-pyrrolo[2,3-d]pyrimidin-2-yl}amino)benzoic acid (0.42 g) was suspended in DMF (3 ml). The suspension was treated with DIPEA (0.84 ml) followed by TBTU (0.48 g) and left for 20 min. Thiomorpholine 1,1-dioxide (27.0 mg, Syntech) was suspended in DMF (0.25 ml) and one twelfth (˜0.25 ml) of the activated ester mixture was added. The reaction mixtures were left at room temperature under nitrogen. The reaction mixture was purified by MDAP, the appropriate fractions combined and the solvent evaporated by vacuum centrifuge. The residue was dissolved in a small amount of methanol and filtered through an aminopropyl cartridge (1 g, pre-conditioned with methanol). The cartridge was washed with methanol and the solvent evaporated from the combined filtrate and washings under vacuum to give the desired product. LC/MS; MH⁺ 468.84, Rt 2.60 min.

### Example 46

### N-Ethyl-N-methyl-4-({4-[(2,2,2-trifluoroethyl)amino]-1H-pyrrolo[2,3-d]pyrimidin-2-yl}amino)benzamide

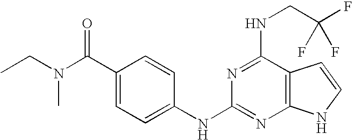

A mixture of 4-({4-[(2,2,2-trifluoroethyl)amino]-1H-pyrrolo[2,3-d]pyrimidin-2-yl}amino)benzoic acid (35.1 mg) and TBTU (35.3 mg) in DMF (0.5 ml) was treated with DIPEA (0.053 ml) and the mixture was stirred vigorously at room temperature for 30 min. N-Ethylmethylamine (0.086 ml) was added and the mixture stirred for 1.25 h. DIPEA (0.026 ml) and TBTU (15.0 mg) were added and the mixture was stirred for 30 min. N-Ethylmethylamine (0.042 ml) was then added and the mixture stirred for a further 1.25 h. The solvent was evaporated under vacuum, the residue dissolved in a small amount of methanol and applied to an SCX-2 cartridge (1 g, pre-conditioned with methanol). The cartridge was eluted with methanol, then with 2M ammonia in methanol. The appropriate fractions were collected and the solvent evaporated under vacuum. The residue was further purified by MDAP to give, after the appropriate fractions were combined and the solvent evaporated under vacuum, the title compound (12 mg). LC/MS; Rt 2.76 min, MH⁺ 393.

### Example 47

### N-(2,2,2-Trifluoroethyl)-4-({4-[(2,2,2-trifluoroethyl)amino]-1H-pyrrolo[2,3-d]pyrimidin-2-yl}amino)benzamide

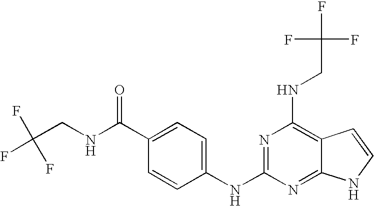

A stirred mixture of 4-amino-N-(2,2,2-trifluoroethyl)benzamide (64.7 mg), 2-chloro-7-[(4-methylphenyl)sulfonyl]-N-(2,2,2-trifluoroethyl)-7H-pyrrolo[2,3-d]pyrimidin-4-amine (100 mg), potassium carbonate (47.8 mg), 2-dicyclohexylphosphino-2′,4′,6′-triisopropyl biphenyl (5.9 mg) and tris(dibenzylideneacetone)dipalladium (0) (13.6 mg) in t-butanol (2.5 ml) was heated in a sealed vial by microwave irradiation at 120° C. for 1 h. The mixture was cooled to room temperature and applied to an SCX-2 SPE cartridge (20 g). The cartridge was washed with methanol and the product eluted with 2M ammonia in methanol. The ammoniacal fractions were collected and the solvent evaporated under vacuum. The residue was treated with IPA (3 ml) and aqueous sodium hydroxide solution (2M, 3 ml) and the mixture was heated at 60° C. overnight. The solvent was evaporated under reduced pressure, the residue was dissolved in methanol and the solution was applied to an SCX-2 cartridge (20 g). The cartridge was washed with methanol and the product eluted with a solution of 2M ammonia in methanol. The basic fractions were combined and the solvent evaporated under vacuum. The residue was purified by MDAP to give, after the appropriate fractions were combined and the solvent evaporated under vacuum, the title compound (60 mg). LC/MS; Rt 2.99 min, MH⁺ 432.86.

### Intermediate 31

### 4-Amino-N-(2,2,2-trifluoroethyl)benzamide

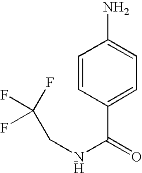

A solution of 4-nitro-N-(2,2,2-trifluoroethyl)benzamide (550 mg) in ethanol (30 ml) was hydrogenated (1 Atm.) over palladium on carbon (10%, 55 mg) overnight. The mixture was filtered through a Celite pad and the residue washed with ethanol. The filtrate was refiltered through Celite and the Celite washed with ethanol. The solvent was evaporated from the combined filtrate and washings under vacuum to give the title compound as a white solid (290 mg). LC/MS; Rt 1.91 min, MH⁺ 219.

### Intermediate 32

### 4-Nitro-N-(2,2,2-trifluoroethyl)benzamide

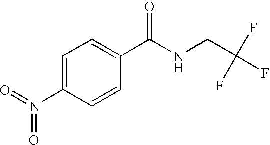

A mixture of 4-nitrobenzoyl chloride (750 mg) and potassium carbonate (606.6 mg) in DCM (40 ml) was treated with 2,2,2-trifluoroethylamine (0.482 ml) and the mixture was stirred at room temperature under nitrogen for 2 h 40 min. Potassium carbonate (606 mg) and 2,2,2-trifluoroethylamine (0.482 ml) were added and the mixture was stirred at room temperature for a further 1.5 h. Water (40 ml) was added and the mixture was stirred vigorously for 15 min. The layers were allowed to separate, the organic phase isolated (hydrophobic frit) and the solvent evaporated under vacuum to give the title compound as a white solid (550 mg). LC/MS: Rt 2.63 min, [M−H]⁻ 247.

### Example 48

### N²-[4-(1-Piperidinylcarbonyl)phenyl]-N⁴-(2,2,2-trifluoroethyl)-1H-pyrrolo[2,3-d]pyrimidine-2,4-diamine

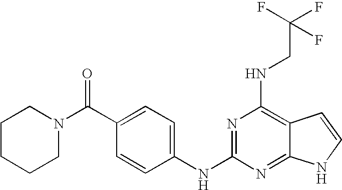

A mixture of 4-(1-piperidinylcarbonyl)aniline (100 mg, Fluorochem), 2-chloro-7-[(4-methylphenyl)sulfonyl]-N-(2,2,2-trifluoroethyl)-7H-pyrrolo[2,3-d]pyrimidin-4-amine (165 mg), potassium carbonate (79 mg), 2-dicyclohexylphosphino-2′,4′,6′-triisopropyl biphenyl (9.8 mg) and tris(dibenzylideneacetone)dipalladium (0) (22.5 mg) in t-butanol (2.5 ml) was heated in a sealed tube by microwave irradiation at 120° C. for 1 h. The mixture was cooled to room temperature and the solvent was evaporated under vacuum. The residue was dissolved in a small amount of methanol and applied to an SCX-2 cartridge (5 g, pre-conditioned with methanol). The cartridge was washed with methanol and the product eluted with 2M ammonia in methanol. The appropriate fractions were collected and the solvent evaporated under vacuum. The residue was dissolved in a small amount of methanol, adsorbed onto Florisil and purified by chromatography on a silica cartridge (70 g) eluting with an ethyl acetate/cyclohexane gradient (0-100%) over 60 min. After combination of the appropriate fractions and evaporation of the solvent under vacuum, the residue was dissolved in a small amount of ether and the solvent was evaporated under vacuum to leave a white solid (194 mg).

The solid was treated with potassium carbonate (340 mg), methanol (2 ml) and water (1 ml) and the mixture was heated at 80° C. overnight. Aqueous sodium hydroxide solution (2M, 1 ml) was added and heating to 80° C. continued for a further 4.5 h. The mixture was cooled to room temperature and was partitioned between ethyl acetate and water. The aqueous phase extracted with ethyl acetate (3×20 ml). The organic phases were combined and evaporated under vacuum. Sodium methoxide in methanol (0.5M, 3 ml) was added to the residue and this stirred mixture was heated at 80° C. for 3 h. The solvent was evaporated under vacuum, the residue dissolved in the minimum amount of methanol and the solution applied to an SCX-2 cartridge (10 g, pre-conditioned with methanol). The cartridge was washed with methanol and the product eluted with 2M ammonia in methanol. The appropriate fractions were combined and the solvent was evaporated under vacuum. The residue was purified by MDAP to give, after the appropriate fractions were combined and the solvent evaporated under vacuum, the title compound (14 mg). LC/MS; Rt 2.96 min, MH⁺ 419.

### Example 49

### N²-[4-(1-Pyrrolidinylcarbonyl)phenyl]-N⁴-(2,2,2-trifluoroethyl)-1H-pyrrolo[2,3-d]pyrimidine-2,4-diamine

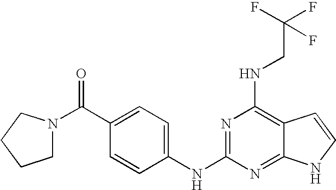

A stirred mixture of 4-(1-pyrrolidinylcarbonyl)aniline (56.4 mg), 2-chloro-7-[(4-methylphenyl)sulfonyl]-N-(2,2,2-trifluoroethyl)-7H-pyrrolo[2,3-d]pyrimidin-4-amine (100 mg), potassium carbonate (48 mg), 2-dicyclohexylphosphino-2′,4′,6′-triisopropyl biphenyl (6 mg) and tris(dibenzylideneacetone)dipalladium (0) (14 mg) in t-butanol (2.5 ml) was heated in a sealed vial by microwave irradiation at 120° C. for 1 h. The mixture was cooled to room temperature and potassium carbonate (24 mg), 2-dicyclohexylphosphino-2′,4′,6′-triisopropyl biphenyl (3 mg) and tris(dibenzylideneacetone)dipalladium (0) (7 mg) were added. The stirred mixture was then heated in a sealed vial by microwave irradiation at 120° C. for 1 h. The mixture was cooled to room temperature and applied to an SCX-2 cartridge (20 g). The cartridge was washed with ethyl acetate, and the product eluted with 2M ammonia in methanol. The appropriate fractions were collected and the solvent evaporated under vacuum. The residue was suspended with IPA (3 ml), treated with aqueous sodium hydroxide solution (2M, 3 ml) and the mixture was heated at 60° C. overnight. The solvent was evaporated under vacuum, the residue dissolved in ethyl acetate and washed twice with hydrochloric acid. The organic phase was reduced to dryness in vacuo. The residue was purified by MDAP, the appropriate fractions combined and the solvent evaporated under vacuum, to give the title compound (17 mg). LC/MS; Rt 2.83 min, MH⁺ 405.

### Intermediate 33

### 4-(1-Pyrrolidinylcarbonyl)aniline

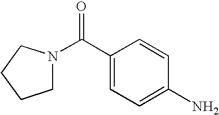

A solution of 1-[(4-nitrophenyl)carbonyl]pyrrolidine (500 mg) in ethanol (30 ml) was hydrogenated (1 Atm.) over palladium on carbon (5%, 50 mg) overnight. The mixture was filtered through Celite, and the catalyst washed twice with ethanol. The solvent was evaporated under vacuum to give the title compound as a white solid (402 mg). LC/MS; Rt 1.86 min, MH⁺ 191.

### Intermediate 34

### 1-[(4-Nitrophenyl)carbonyl]pyrrolidine

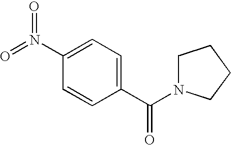

A mixture of 4-nitrobenzoyl chloride (750 mg) in DCM (50 ml) was treated with pyrrolidine (1.66 ml) and the mixture was stirred at room temperature under nitrogen for 5 h. Hydrochloric acid (1M, 50 ml) was added and the mixture was stirred vigorously for 20 min. The layers were separated and the organic phase washed with sodium hydrogen carbonate solution (50 ml), then water and reduced to dryness under vacuum to give the title compound (500 mg). LC/MS; Rt 2.54 min, MH⁺ 221.

### Example 50

### N²-[4-(1-Azetidinylcarbonyl)phenyl]-N⁴-(2,2,2-trifluoroethyl)-1H-pyrrolo[2,3-d]pyrimidine-2,4-diamine

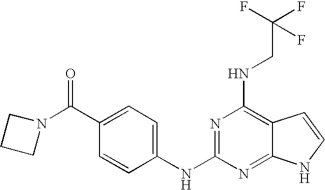

A stirred mixture of 4-(1-azetidinylcarbonyl)aniline (52.2 mg), 2-chloro-7-[(4-methylphenyl)sulfonyl]-N-(2,2,2-trifluoroethyl)-7H-pyrrolo[2,3-d]pyrimidin-4-amine (100 mg), potassium carbonate (47.8 mg), 2-dicyclohexylphosphino-2′,4′,6′-triisopropyl biphenyl (6 mg) and tris(dibenzylideneacetone)dipalladium (0) (13.6 mg) in t-butanol (2.5 ml) was heated in a sealed vial by microwave irradiation at 120° C. for 1 h. The mixture was cooled to room temperature and applied to an SCX-2 SPE cartridge (20 g). The cartridge was washed with methanol, ethyl acetate, and the product eluted with 2M ammonia in methanol. The basic fractions were collected and the solvent evaporated under vacuum. The residue was suspended in IPA (3 ml) and treated with aqueous sodium hydroxide solution (2M, 3 ml) and the mixture was heated at 60° C. overnight. The solvent was evaporated under reduced pressure. DCM was added to the residue and the insoluble material was isolated by filtration. The solid was dissolved in methanol (30 ml) and the solvent was evaporated under vacuum. The residue was dissolved in chloroform, the solution applied to an aminopropyl SPE (10 g) and eluted with chloroform, ethyl acetate and methanol. The chloroform fractions were combined and the solvent evaporated under vacuum. The residue was purified by MDAP to give, after the appropriate fractions were combined and the solvent evaporated under vacuum, the title compound (6 mg). LC/MS; Rt 2.73 min, MH⁺ 391.

### Intermediate 35

### 4-(1-Azetidinylcarbonyl)aniline

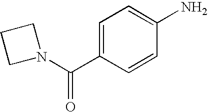

A solution of 1-[(4-nitrophenyl)carbonyl]azetidine (463 mg) in ethanol (30 ml) was hydrogenated over palladium on carbon (46.3 mg) overnight. The mixture was filtered through a Celite pad, which was washed twice with ethanol. The solvent was evaporated under vacuum to give the title compound as a yellow solid. (340 mg). LC/MS; Rt 1.72 min, MH⁺ 177.

### Intermediate 36

### 1-[(4-Nitrophenyl)carbonyl]azetidine

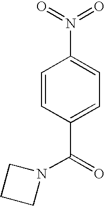

A mixture of 4-nitrobenzoyl chloride (750 mg) and potassium carbonate (607 mg) in DCM (50 ml) was treated with azetidine (0.408 ml) and the mixture was stirred at room temperature under nitrogen for 4 h 20 min. Potassium carbonate (606 mg) and azetidine (0.408 ml) were added and the mixture was stirred at room temperature for a further 40 min. Water (50 ml) was added and the mixture was stirred vigorously for 15 min. The layers were allowed to separate and the organic phase was isolated (hydrophobic frit). The solvent was evaporated under vacuum to give the title compound as a yellow solid (463 mg). LC/MS; Rt 2.41 min, MH⁺ 207.

### Example 51

### N-Methyl-N-propyl-4-({4-[(2,2,2-trifluoroethyl)amino]-1H-pyrrolo[2,3-d]pyrimidin-2-yl}amino)benzamide

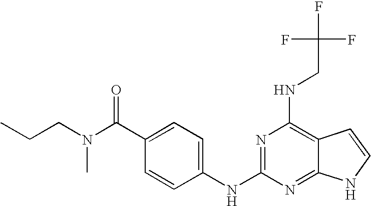

A stirred mixture of 4-amino-N-methyl-N-propylbenzamide (57 mg), 2-chloro-7-[(4-methylphenyl)sulfonyl]-N-(2,2,2-trifluoroethyl)-7H-pyrrolo[2,3-d]pyrimidin-4-amine (100 mg), potassium carbonate (48 mg), 2-dicyclohexylphosphino-2′,4′,6′-triisopropyl biphenyl (6 mg) and tris(dibenzylideneacetone)dipalladium (0) (14 mg) in t-butanol (2.5 ml) was heated in a sealed vial by microwave irradiation at 120° C. for 1 h. The cooled reaction was applied to a pre-conditioned SCX-2 cartridge (20 g). The cartridge was washed with methanol and the product eluted with 2M ammonia in methanol. The ammoniacal fractions were collected and the solvent evaporated under vacuum. The residue was treated with potassium carbonate (423.5 mg), methanol (2 ml) and water (1 ml) and the stirred mixture was heated at 80° C. overnight. Water (5 ml) was added and the precipitate was isolated by filtration. The filtrate was extracted with ethyl acetate (30 ml), the organic phase dried (hydrophobic frit) and the solvent evaporated under vacuum. The residue and the precipitate were dissolved in methanol, combined and reduced to dryness in vacuo. The resulting residue was purified by MDAP to give, after the appropriate fractions were combined and the solvent evaporated under vacuum, the title compound (19 mg). LC/MS; Rt 2.91 min, MH⁺ 406.9.

### Intermediate 37

### 4-Amino-N-methyl-N-propyl benzamide

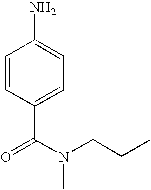

A solution of N-methyl-4-nitro-N-propylbenzamide (330 mg) in ethanol (15 ml) was hydrogenated (1 Atm.) over palladium on carbon (10%, 15.3 mg) overnight. The mixture was filtered through a Celite pad which was then washed with ethanol. The solvent was evaporated under vacuum to give the title compound (260 mg). LC/MS; Rt 2.02 min, MH⁺ 193.

### Intermediate 38

### N-Methyl-4-nitro-N-propylbenzamide

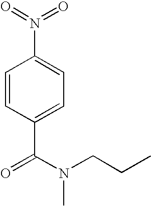

A mixture of 4-nitrobenzoyl chloride (750 mg), N-methylpropylamine (0.622 ml) and potassium carbonate (836 mg) in DCM (50 ml) was stirred at room temperature under nitrogen overnight. Hydrochloric acid (1M, 50 ml) was added and the mixture was stirred for 15 min, the phases separated, the organic phase was washed with water (100 ml) and dried (hydrophobic frit). Evaporation of the solvent in vacuo gave a residue which was dissolved in ethyl acetate (25 ml) and was washed with sodium hydrogencarbonate solution (50 ml). The organic phase was collected through a hydrophobic frit and the solvent was evaporated under vacuum to give the title compound (330 mg). LC/MS; Rt 2.58 min, MH⁺ 223.

### Example 52

### N-propyl-4-({4-[(2,2,2-trifluoroethyl)amino]-1H-pyrrolo[2,3-d]pyrimidin-2-yl}amino)benzamide 4-methylbenzenesulfonate

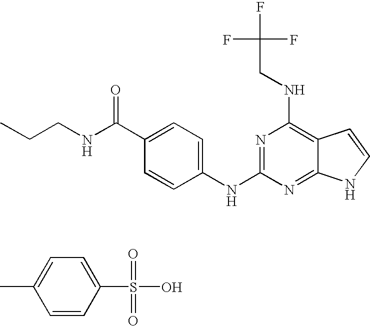

N-Propyl-4-({4-[(2,2,2-trifluoroethyl)amino]-1H-pyrrolo[2,3-d]pyrimidin-2-yl}amino)benzamide (350 mg) was dissolved with heating and sonication in THF (7 ml). p-Toluenesulphonic acid hydrate (162 mg) was dissolved with heating in THF (1 ml) and the resulting solution added to the N-propyl-4-({4-[(2,2,2-trifluoroethyl)amino]-1H-pyrrolo[2,3-d]pyrimidin-2-yl}amino)benzamide. The mixture was warmed gently to give a solution and then allowed to cool to room temperature. The mixture was rewarmed to 40° C., allowed to cool and the heating cooling cycle repeated (×2). The mixture was left at room temperature over the weekend, the white solid isolated by filtration, washed with THF (1 ml) and sucked dry on the sinter. Solid further dried under vacuum at ˜40° C. for 2 h to give the title compound as a white solid (425 mg). NMR; [D₆-DMSO] δH 11.46, (1H, b), 9.44, (1H, b), 8.47, (1H, b), 8.28, (1H, t), 7.79, (4H, s), 7.48, (2H, d), 7.11, (2H, d), 6.96, (1H, m), 6.60, (1H, m), 4.40, (2H, m), 3.21, (2H, q), 2.29, (3H, s), 1.53, (2H, m), 0.89, (3H, t).

Biological Test Methods

Compounds of the invention may be tested for in vitro activity in accordance with the following assays:

1. Enzyme Assay—Time-Resolved Fluorescence Resonance Energy Transfer Kinase Assay

Recombinant human Syk was expressed as a His-tagged protein*. The activity of Syk was assessed using a time-resolved fluorescence resonance energy transfer (TR-FRET) assay.

Version A—3 μl of substrate reagent containing biotinylated peptide, Biotin-AAAEEIYGEI (0.5 μM final), ATP (30 μM final) and MgCl₂ (10 mM final) in HEPES pH 7.4, (40 mM final), were added to wells containing 0.2 μl of various concentrations of compound or DMSO vehicle (3.3% final) in Greiner low volume 384 well black plate. The reaction was initiated by the addition of 3 μl of Syk (20 nM final) in HEPES pH 7.4 (40 mM final). The reaction was incubated for 40 min at room temperature, then terminated by the addition of 3 μl of read reagent containing 60 mM EDTA, 150 mM NaCl, 50 nM Streptavidin APC (Prozyme, San Leandro, Calif., USA), 0.5 nM antiphosphotyrosine antibody labelled with W-1024 europium chelate (Wallac OY, Turku, Finland) in 40 mM HEPES pH 7.4, 0.03% BSA. The reaction was further incubated for 60 min at room temperature. The degree of phosphorylation of Biotin-AAAEEIYGEI was measured using a BMG Rubystar plate reader (BMG LabTechnologies Ltd, Aylesbury, UK) as a ratio of specific 665 nm energy transfer signal to reference europium 620 nm signal.

Version B—Syk was pre-activated at room temperature for 30 mins in the presence of 16.6 mM MgCl₂, 8.3 mM ATP and then diluted to 4 nM in 40 mM Hepes pH 7.4, 0.01% BSA. 3 μl of substrate reagent containing biotinylated peptide, Biotin-AAAEEIYGEI (0.5 μM final), ATP (30 μM final) and MgCl₂ (10 mM final) in 40 mM HEPES pH 7.4, 0.01% BSA, were added to wells containing 0.1 μl of various concentrations of compound or DMSO vehicle (1.7% final) in Greiner low volume 384 well black plate. The reaction was initiated by the addition of 3 μl of diluted Syk (2 nM final). The reaction was incubated for 60 min at room temperature, then terminated by the addition of 3 μl of read reagent containing 60 mM EDTA, 150 mM NaCl, 50 nM Streptavidin APC (Prozyme, San Leandro, Calif., USA), 0.5 nM antiphosphotyrosine antibody labelled with W-1024 europium chelate (Wallac OY, Turku, Finland) in 40 mM HEPES pH 7.4, 0.03% BSA. The reaction was further incubated for 45 min at room temperature. The degree of phosphorylation of Biotin-AAAEEIYGEI was measured using a BMG Rubystar plate reader (BMG LabTechnologies Ltd, Aylesbury, UK) as a ratio of specific 665 nm energy transfer signal to reference europium 620 nm signal.

Compounds according to the present invention were assayed in this, or a similar Time-resolved fluorescence resonance energy transfer kinase assay, and gave IC₅₀ values less than 10 μM.

Preparation of Recombinant Human Full Length Spleen Tyrosine Kinase (Syk) Syk

Full length human Syk was expressed with a 6H is tag on the N-terminal using the baculovirus system (Invitrogen, Paisley, Scotland). The cells were disrupted by dounce homogenisation, the debris removed by centrifugation and the lysate contacted with NiNTA Superflow (Qiagen, Crawley, UK). The NiNTA was packed into a column and eluted using 10 column volumes each of buffer (20 mM Tris pH8.0, 300 mM NaCl, 10 mM βMcEtOH, 10% glycerol), buffer+1M NaCl, buffer+20 mM Imidazole and buffer+300 mM imidazole. The 300 mM Imidazole fractions were pooled buffer exchanged using G25M (Amersham Biosciences, Buckinghamshire, UK) into 20 mM MES pH 6.0, 20 mM NaCl, 10 mM βMcEtOH, 10% glycerol. The buffer exchanged 6His-Syk was loaded onto a Source15S column (Amersham Biosciences, Buckinghamshire, UK) and the column eluted using a NaCl gradient 0-500 mM over 50 column volumes. The 6His-Syk containing fractions were pooled and concentrated by ultra-filtration. The identity of 6His-Syk was confirmed by peptide mass finger printing and intact LC-MS.

2. Whole Cell Assay—cFms Assay

Principle of the Assay

Cells of the mouse fibroblast cell line NIH-3T3 are stably transfected with a cFms-SYK chimera. Addition of the ligand (MCSF) produces dimerisation of the chimera resulting in autophosphorylation of the SYK kinase domain. Following cell lysis phosphorylated SYK is detected by ELISA.

Stimulation of cFms-SYK Cells with MCSF Version A

Cells are plated at a density of 1×10⁵/well in a volume of 200 μl growth medium (DMEM containing 10% heat inactivated foetal calf serum, 1% L-glutamine, 400 μg/ml geneticin and 400 μg/ml zeocin) in 96 well Collagen 1 coated tissue culture plates. Following incubation at 37° C., 10% CO₂, for 20 h, the cell supernatant is removed and replaced with 200 μl DMEM containing 1% penicillin/streptomycin (serum free DMEM). The cells are incubated for one hour under the conditions described above. The medium is removed, 50 μl appropriately diluted compound solution added and the plate incubated for a further hour. Cells are stimulated with 25 μl MCSF (0.66 μg/ml final) for 20 min at 37° C. After removal of the supernatant, the cells are washed with cold PBS and lysed with 100 μl lysis buffer for 4 h at 4° C.

Stimulation of cFms-SYK Cells with MCSF Version B

Cells are plated at a density of 1×10⁵/well in a volume of 200 μl growth medium (DMEM containing 10% heat inactivated foetal calf serum, 1% L-glutamine, 400 μg/ml geneticin and 400 μg/ml zeocin) in 96 well Collagen 1 coated tissue culture plates. Following incubation at 37° C., 10% CO₂, for 20 h the cell supernatant is removed and 50 μl appropriately diluted compound solution added and the plate incubated for an hour. Cells are stimulated with 25 μl MCSF (0.66 μg/ml final) for 20 min at 37° C. After removal of the supernatant, the cells are washed with cold PBS and lysed with 100 μl lysis buffer for 4 h at 4° C.

cFms ELISA

85 μl cell lysate is transferred to a 96 well ELISA plate coated with goat anti human M-CSF R capture antibody and incubated for 16 hours at 4° C. The plate is washed and a biotinylated anti-phosphotyrosine detection antibody added (100 μl/well) for 2 h at room temperature. This is removed and replaced with 100 μl Streptavidin-HRP for 30 min. Captured phosphorylated SYK is visualised using 100 μl TMB substrate. The reaction is terminated with 50 μl 1M sulphuric acid and the absorbance measured at 450 nm.

Compound Preparation

Compound is prepared as a 10 mM stock in DMSO and a dilution series prepared in DMSO using 9 successive 5-fold dilutions. This dilution series is diluted a further 1:333 with serum free DMEM to give the concentration range to be tested of 1×10⁻⁵ to 1.54×10⁻¹¹M. Compound dilutions are prepared using the Biomek 2000 or Biomek Nx automated robotic pipetting systems.

3. B Cell Proliferation Assay

Background

The population of B cells observed in this assay are the naïve mature IgM/IgD expressing population. These form at least 70% of the purified B cell population (the rest being isotype switched memory B cells) and are the only cells that proliferate as the cells are stimulated with anti-IgM.

Anti-IgM drives signalling through the B cell receptor which is Syk dependant. Proliferation is a functional measure of B cell signalling that can be measured by observing the incorporation of tritiated methyl thymidine into the cells.

Protocol

Purified human tonsillar B cells are resuspended in Buckleys* medium at a concentration of 1.25×10⁶ ml.

160 μl of cells re-suspended in Buckley's medium is added to the compound and control wells of a 96 well plate. The control wells are located on column 11 and 12 of the 96 well plate. The background wells are located in column 12 and 20 μl of 10 μM control is added to provide an appropriate background control. 20 μl of 1% DMSO is added to the wells in column 11 for the stimulated control.

The compound titrations are located between columns 1 and 10. Three compounds are run in duplicate on each plate and row A and B are used for the control compound titration.

The final concentration of DMSO is 0.1% in the assay. The cells are left for 45 min, after 45 min the proliferative stimulus is added to the first 11 wells of the 96 well plate and 20 μl of medium is added to column 12. F(ab′)2 fragments of a polyclonal goat anti-sera raised to human IgM is used at a final concentration of 15 μg/ml to stimulate the cells. (Biosource. Cat no: AMI 4601).

Tritiated methyl thymidine is added to the cells at a concentration of 1 μCi per well. (Amersham, TRK 758). The radioactivity is added 65 hours after the initial stimulus and is left on the cells for 6 to 8 hours. After pulsing with methyl thymidine the cells are harvested on a Skatron 96 well cell harvester onto glass fibre mats. Once these have dried these are counted on a Wallac 1450 Microbeta scintillation counter.

Data is downloaded as an XL file and IC50's determined using Activity base.
Buckleys Medium: 450 ml Iscoves (Sigma 13390), 50 ml FCS, 2.5 g BSA, 5 ml Pen/strep, 5 ml Glutamine (200 mM), 500 μl Apo transferrin (50 mg/ml) Sigma (T 1147), 100 μl Bovine Insulin (10 mg/ml) Sigma (I 1882).
   Compound Preparation

Compound is prepared as a 10 mM stock in DMSO and a dilution series prepared in DMSO using 9 successive 3-fold dilutions. This dilution series is diluted a further 1:100 with Buckleys medium to give the concentration range to be tested of 100 μM to 5 nM. This is added as 20 μl to 96 well plates in duplicate to generate two IC50's for each compound tested. Each plate is run in the presence of a control compound, which acts as an internal standard.

4. LAD2 Assay

Principle of the Assay

LAD2 is a stem cell factor (SCF)-dependent human mast cell line that was established by the NIH from bone marrow aspirates from a patient with mast cell sarcoma/leukaemia. LAD2 cells resemble CD34+-derived human mast cells and express functional FcεRI. The FcεRI is up-regulated in the presence of IL-4, SCF and IgE, subsequent cross linking of cell-bound IgE results in degranulation which can be measured as hexosaminidase release.

Priming LAD2 Cells to Up-Regulate FcεR1

LAD2 cells are re-suspended at 1×10⁵/ml in complete stem pro-34SFM (Gibco Cat 10640-019 media containing Stem Pro-34 nutrient supplement (1:40), glutamine (2 mM), penicillin (100 μg/ml), streptomycin (100 μg/ml)) with additional supplements of human recombinant SCF (100 ng/ml; R&D systems), human recombinant Interleukin-4 (6 ng/ml; R&D Systems) and IgE (100 μg/ml; Calbiochem). Cells are then maintained for 5 days at 37° C., 5% CO2 in a humidified atmosphere.

Compound Preparation

Compounds are titrated from a 2 mM stock in 100% DMSO to give 9 successive 1:3 dilutions (V 96-well Nunc; Biomek 2000). From this master plate 3 μl is dispensed into a daughter plate (flat 96-well NuncBiomek Fx) which is then diluted 1:40 in RPMI with 2 mM glutamine, and 20 μl of the diluted compound transferred into the Greiner cell plate. Therefore the final compound concentration range is 1×10⁻⁵M to 5×10⁻¹⁰M in a constant 0.5% DMSO. Control wells are treated with 0.5% DMSO.

Activation of LAD2 Cells with Anti-IgE Version A

Primed LAD2 cells are centrifuged (300 g, 5 min), the supernatant discarded and the cell pellet re-suspended at 1×10⁴ cells/ml in RPMI supplemented with glutamine (2 mM). Following a further centrifugation (300 g, 5 min) the cells are re-suspended in fresh RPMI with glutamine (2 mM), adjusted to a density of 2.85×10⁵/ml, and pipetted into sterile V-well plates (70 μl/well; Greiner) containing 20 μl diluted compound (prepared as detailed above). Cells are then incubated for 1 h (37° C., 5% CO₂ in a humidified atmosphere) before activating with a sub-maximal concentration of anti-IgE (10 μl volume to give a final assay dilution of 1:2700; Sigma). Following a 40 min incubation (37° C., 5% CO₂ in a humidified atmosphere), plates are centrifuged (1200 g, 10 min, 4° C.) and the supernatant removed for hexosaminidase assay. The cell pellet is lysed in 100 μl/well triton-X (0.5% in RPMI 2 mM glutamine) at 37° C. for 30 min.

Activation of LAD2 Cells with Anti-IgE Version B

Primed LAD2 cells are centrifuged (400 g, 5 min), the supernatant discarded and the cell pellet re-suspended at 1×10⁴ cells/ml in RPMI supplemented with glutamine (2 mM). Following a further centrifugation (400 g, 5 min) the cells are re-suspended in fresh RPMI with glutamine (2 mM), adjusted to a density of 5.7×10⁵/ml, and pipetted into sterile V-well plates (70 μl/well; Greiner) containing 20 μl diluted compound (prepared as detailed above). Cells are then incubated for 1 h (37° C., 5% CO₂ in a humidified atmosphere) before activating with a sub-maximal concentration of anti-IgE (10 μl volume to give a final assay dilution of 1:2700; Sigma). Following a 40 min incubation (37° C., 5% CO₂ in a humidified atmosphere), plates are centrifuged (1200 g, 10 min, 4° C.) and the supernatant removed for hexosaminidase assay. The cell pellet is lysed in 100 μl/well triton-X (0.5% in RPMI 2 mM glutamine) at 37° C. for 30 min.

Beta-Hexosaminidase Assay

Beta-hexosaminidase activity is measured by the conversion of 4-methylumbelliferyl N-acetyl-ε-D glucosaminide (Sigma) to a fluorescent product. Supernatant or lysate (25 μl) is incubated with an equal volume of 4-methylumbelliferyl N-acetyl-ε-D glucosaminide (500 μM in 0.2M sodium citrate buffer, pH 4.5) in black 96-well plate (Nunc) for 1 h at 37° C. The reaction is then terminated by addition of Trizma pH9 (90 μl) and the fluorescent product measured using excitation 356 nm and emission 450 nm (Tecan Safire)

A useful screening strategy comprises assay 1 (enzyme assay (pKi), assay 2 and then assay 3 (B Cell Proliferation) or assay 4 (LAD2).

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A compound or a pharmaceutically acceptable salt or solvate thereof which is:
N-Propyl-4-({4-[(2,2,2-trifluoroethyl)amino]-1H-pyrrolo[2,3-d]pyrimidin-2-yl}amino)benzamide.

2. A pharmaceutical composition comprising a compound according to claim 1 or a pharmaceutically acceptable salt or solvate, thereof and one or more of pharmaceutically acceptable carriers, diluents and excipients.

3. A method of treating a disease or condition mediated by inappropriate Syk activity in a mammal comprising administering to said mammal a compound of formula (I) according to claim 1 or a salt or solvate thereof.

4. A method as claimed in claim 3 in which the disease or condition mediated by inappropriate Syk activity is rheumatoid arthritis.

5. A method as claimed in claim 3 in which the disease or condition mediated by inappropriate Syk activity is allergic rhinitis.

6. A method as claimed in claim 3 in which the disease or condition mediated by inappropriate Syk activity is chronic obstructive pulmonary disease (COPD).

7. A method as claimed in claim 3 in which the disease or condition mediated by inappropriate Syk activity is adult respiratory distress syndrome (ARDs).
